(19) United States
(12) Patent Application Publication
(10) Pub. No.: US 2026/0083976 A1
(43) Pub. Date: Mar. 26, 2026

(54) PLASMA-ACTIVATED COMPOSITIONS AND METHODS OF USE THEREOF

(71) Applicant: Nova Plasma Ltd., Hevel Megiddo (IL)

(72) Inventors: Amnon LAM, Kibbutz Givat Oz (IL); Aviad HARHOL, Ramat Hasharon (IL); Betsalel RECHAV, Ness Ziona (IL); Chen PORAT, Kiryat Tivon (IL); Pierre SHARVIT, Emek Hefer (IL); Raz GIBRALTAR, Haifa (IL)

(21) Appl. No.: 19/112,388

(22) PCT Filed: Sep. 14, 2023
(86) PCT No.: PCT/IL2023/051007

§ 371 Date: Mar. 17, 2025

Related U.S. Application Data
(60) Provisional application No. 63/376,090, filed on Sep. 18, 2022.

(51) Int. Cl. A61N 1/44; H05H 1/48

(52) U.S. Cl. CPC A61N 1/44; H05H 1/48; H05H 2245/20

(57) ABSTRACT

The present invention, in some aspects thereof, provides a plasma-activated liquid composition capable of in-situ gelation upon a stimulus, e.g., a change in temperature or a change in pH or induced by light. The plasma-activated liquid composition is useful in the prophylaxis, treatment, and post-treatment of malignancies as well as in treating various infections. Some aspects of the invention further provide devices, systems and methods for plasma activating a liquid medium, and for providing plasma activated medium from a liquid composition as described above.

### FIELD OF INVENTION

The present invention relates to plasma-activated compositions and related systems, apparatuses and methods of use thereof in the prophylaxis, treatment, and post-treatment of various diseases and disorders, in particular infections and cancer.

### BACKGROUND

Low temperature plasma has been gaining interest in recent years due to its use in a variety of medical applications including wound healing, disinfection and oncology to name a few. The mechanism of action of plasma therapy has been attributed to reactive oxygen and nitrogen species (ROS and RNS respectively, and collectively RONS) generated by the plasma, said species interact with cells to induce subsequent reactions within the cells that can trigger cell-signaling cascades (Laroussi, Plasma 2018, 1(1), 47-60; https://doi.org/10.3390/plasma1010005). In order to ensure effective treatment, a close contact between the cells and the RONS is required. However, even when local delivery of the plasma is afforded by various apparatuses and devices, gaseous plasma often rapidly escapes from the site of action thus lowering therapeutic efficacy.

Plasma-activated media (PAM) have also been used for medical treatments. A medium may be plasma-activated by exposing the medium to a gaseous phase which is by itself being excited to plasma, e.g., by an electromagnetic (EM) field. ROS and RNS which are generated in the gaseous plasma are dissolved in the medium thereby rendering the medium plasma-activated. Advantageously, plasma activated media are most often liquids—typically saline or other aqueous solutions—because the diffusion of activated species in liquids is much faster than in solids, and activation duration may consequently be much shorter.

WO 2015/123720 described a plasma treatment method comprising: providing a plasma source and a screen comprising a hydrogel and positioning the screen between the plasma source and a surface of a target to be treated with the plasma such that substantially all of the plasma from the plasma source passes through the screen prior to contacting the surface of the target and the screen reduces the concentration of one or more species from the plasma; and/or contacting a surface of a target to be treated with the gel composition comprising a gel forming material and a liquid phase comprising plasma activated liquid.

Labay et al. (Scientific Reports 2019, 9, 16160; https/do&iorg/10.1038/A41598-019-52673-w) investigated the generation of RONS in alginate hydrogels by comparing two atmospheric pressure plasma jets, namely KINPen and a helium needle, at a range of plasma treatment conditions. The hydrogels showed capacity for sustained release of the RONS with cytotoxic potential towards bone cancer cells.

Labay et al. (ACS Appl Mater Interfaces 2020, 12(42), 47256-47269; doi: 10.1021/acsami.0c12930) investigated the formation and release of RONS generated in 2% gelatin. In vitro studies on the sarcoma osteogenic (SaOS-2) cell line exposed to plasma-treated gelatin led to time-dependent increasing cytotoxicity with the longer plasma treatment time of gelatin. While the SaOS-2 cell viability decreased to 12%-23% after 72 h for cells exposed to 3 min of treated gelatin, the viability of healthy cells (hMSC) was preserved (˜90%), establishing the selectivity of the plasma-treated gelatin on cancer cells.

WO 2021/255179 described a composition comprising a polymer aqueous solution, a bioceramic material and reactive oxygen and nitrogen species (RONS) and its use for the treatment of bone cancer and/or bone tissue regeneration.

### SUMMARY

Due to the short lifetime of the RONS created by a cold plasma apparatus in the gaseous phase, there is a great unmet need for compositions with increased residence time at the site of action to induce the effective release of RONS for the treatment of various diseases and disorders.

The present invention provides a composition in the form of a liquid that has been activated by plasma to generate RONS, the composition comprises a synthetic biocompatible polymer capable of in-situ gelation in response to a stimulus. The composition is useful in the prophylaxis, treatment, and post-treatment of cancer, and the treatment of viral, bacterial, yeast, mold, and fungal infections as well as other medical conditions.

The present invention is based, in part, on the unexpected finding that administration of a plasma-activated liquid that undergoes in-situ phase transition to form a hydrogel has therapeutic efficacy in treating pre-malignant lesions, as well as in the prevention of tumor recurrence following tumor excision. The composition of the present invention can also be used for the treatment of various types of cancer including, in particular, cancer in soft tissue. Within the scope of the present invention is the treatment of infections by topically applying a plasma-activated liquid composition whereby due to in-situ gel formation provides prolonged residence time of active species at the site of action with improved therapeutic efficacy.

According to a first aspect, there is provided a liquid composition comprising a plurality of reactive oxygen and nitrogen species generated by plasma and a synthetic biocompatible polymer, wherein the composition undergoes a phase transition to a gel form upon a stimulus.

In one embodiment, the plurality of reactive oxygen and nitrogen species generated by plasma comprise at least one of H₂O₂, OH*, HO₂*, O₂⁻, O₃, NO*, NO₂⁻, NO₃⁻, and ONOO*. Each possibility represents a separate embodiment.

In other embodiments, the synthetic biocompatible polymer comprises polyethylene glycol (PEG), polypropylene glycol (PPG), poly(meth)acrylic acid, poly (meth)acrylate or a combination thereof. Each possibility represents a separate embodiment. In particular embodiments, the synthetic biocompatible polymer comprises a poloxamer. In additional embodiments, the synthetic biocompatible polymer comprises a poloxamer selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, or a mixture thereof. Each possibility represents a separate embodiment.

In particular embodiments, the synthetic biocompatible polymer comprises a poloxamer selected from the group consisting of poloxamer 407, poloxamer 188, and poloxamer 338, or a mixture thereof. Each possibility represents a separate embodiment. In specific embodiments, the synthetic biocompatible polymer may comprise a mixture of poloxamers. In particular embodiments, the synthetic biocompatible polymer comprises poloxamer 407 or a mixture of poloxamer 407 and poloxamer 188. In further embodiments, the synthetic biocompatible polymer comprises a mixture of poloxamer 407 and poloxamer 188 at a weight ratio of 10:1 to 1:1, including all iterations of ratios within the specified range. In additional embodiments, the synthetic biocompatible polymer comprises a mixture of poloxamer 407 and poloxamer 188 at a weight ratio of 6:1 to 1:1, including all iterations of ratios within the specified range. In other embodiments, the synthetic biocompatible polymer comprises a mixture of poloxamer 338 and poloxamer 188. In specific embodiments, the synthetic biocompatible polymer comprises a mixture of poloxamer 338 and poloxamer 188 at a weight ratio of 10:1 to 1:1, including all iterations of ratios within the specified range. In yet other specific embodiments, the synthetic biocompatible polymer comprises a mixture of poloxamer 338 and poloxamer 188 at a weight ratio of 6:1 to 1:1, including all iterations of ratios within the specified range.

In various embodiments, the stimulus comprises a change in temperature. In other embodiments, the stimulus comprises a change in pH. In yet other embodiments, the stimulus includes light-induced cross-linking.

In further embodiments, the composition further comprises a fluid medium. In one embodiment, the fluid medium comprises a buffering or pH adjusting agent. In various embodiments, the buffering or pH adjusting agent is selected from the group consisting of 2-amino-2-hydroxymethyl-1,3-propanediol (Tris), 2-[bis(2-hydroxyethyl)imino]-2-(hydroxymethyl)-1,3-propanediol (bis-Tris), 4-morpholine ethane sulfonic acid (MES) buffer, ammonium chloride, bicine, tricine, sodium phosphate monobasic, sodium phosphate dibasic, sodium carbonate, sodium bicarbonate, sodium acetate, sodium phosphate, glutamic acid, citrate buffer, histidine buffer, Dulbecco's phosphate-buffered saline, 4-(2-hydroxyethyl)-1-piperazineethanesulfonic acid (HEPES), methoxypsoralen (MOPS), N-cyclohexyl-3-aminopropanesulfonic acid (CAPS), N-cyclohexyl-2-hydroxyl-3-aminopropanesulfonic acid (CAPSO), N-Cyclohexyl-2-aminoethanesulfonic acid (CHES), 3-[4-(2-Hydroxyethyl)-1-piperazinyl]propanesulfonic acid (HEPPS), phosphate-buffered saline, tris-buffered saline, Hank's solution, and Ringer's solution, and a mixture or combination thereof. Each possibility represents a separate embodiment. In further embodiments, the composition has a pH in the range of about 5.0 to about 7.5, including each value within the specified range.

In various embodiments, the fluid medium comprises a solvent. In particular embodiments, the solvent is an aqueous solvent. In specific embodiments, the fluid medium comprises water.

In certain embodiments, the composition disclosed herein is useful in treating a pre-malignant lesion. Thus, in accordance with these embodiments there is provided a method of treating a pre-malignant lesion in a subject in need thereof, the method comprising the step of contacting the lesion with a therapeutically effective amount of the liquid composition disclosed herein or a therapeutically effective amount of the composition disclosed herein in a gel form.

In other embodiments, the composition disclosed herein is useful in treating a tumor. Thus, in accordance with these embodiments there is provided a method of treating a tumor in a subject in need thereof, the method comprising the step of contacting the tumor with a therapeutically effective amount of the liquid composition disclosed herein or a therapeutically effective amount of the composition disclosed herein in a gel form.

In further embodiments, the composition disclosed herein is useful in preventing or delaying tumor recurrence following tumor excision. Thus, in accordance with these embodiments there is provided a method of preventing or delaying tumor recurrence following tumor excision in a subject in need thereof, the method comprising the step of contacting the remaining tissue that surrounded the tumor with a therapeutically effective amount of the liquid composition disclosed herein or a therapeutically effective amount of the composition disclosed herein in a gel form.

In some embodiments, the composition disclosed herein is useful in treating a viral, a bacterial, a yeast, a mold, or a fungal infection. Each possibility represents a separate embodiment. Thus, in accordance with these embodiments, there is provided a method of treating an infection selected from a bacterial, a viral, a yeast, a mold, and a fungal infection in a subject in need thereof, the method comprising the step of topically administering to the subject a therapeutically effective amount of the liquid composition disclosed herein or a therapeutically effective amount of the composition disclosed herein in a gel form.

According to a further aspect of the invention there is provided a system for providing plasma activated medium (PAM) for medical use. The system comprises a vessel configured to contain a liquid medium. The system further comprises an electrode electrically associated with a high voltage power source, the electrode being configured to apply a plasma generating electromagnetic field within the vessel. The system further comprises an actuator configured to cause agitation of the liquid medium inside the vessel. And the system yet further comprises a chiller having a cold portion configured to thermally couple with the liquid medium inside the vessel. According to aspects of the invention, the system is configured to generate plasma at atmospheric pressure inside the vessel while the liquid medium is agitated and maintained at a temperature below room temperature.

It is noted that the term ‘chiller’ is used here in a wide sense and refers to any type of a cooling device, cooling system or cooling method.

According to some embodiments the liquid medium is a liquid composition comprising a biocompatible polymer, the liquid composition being capable of undergoing a phase transition to a gel form upon a stimulus, as described above. According to some embodiments the biocompatible polymer is synthetic.

According to another aspect of the invention there is further provided a transportable closed vessel. The closed vessel comprises a liquid medium stored in the vessel, intended for medical use, and an electrode electrically associated with a HV connector. The electrode is configured to apply a plasma-generating EM field inside the vessel upon receiving high voltage via the HV connector. The vessel is biologically sealed so as to prevent penetration of bacteria or viruses into the vessel. According to some embodiments, the vessel is hermetically sealed.

According to some embodiments, the liquid medium is sterile. According to some embodiments, the liquid medium comprises a biocompatible polymer, forming a liquid composition capable of undergoing a phase transition to a gel form upon a stimulus, as described above.

According to an aspect of the invention there is further provided a portable, passive chiller configured to chill a vessel The passive chiller comprises an ice-pack having at least one chamber containing a coolant. The coolant preferably has a freezing temperature between −10 degC. and +5 degC. The at least one chamber of the ice-pack is shaped to surround a slot in the ice-pack, wherein the slot is configured and dimensioned to house the vessel therein.

According to a further aspect of the invention there is provided a foam comprising the reactive species H₂O₂ and NO₂ and at least one of the reactive species OH*, HO₂*, O₂⁻, O₃, NO*, NO₃⁻, and ONOO*. According to some embodiments the foam is made from liquid composition as described above that was plasma activated and transformed to foam by intense agitation.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1C depict schematically an embodiment of a capsule configured to contain a liquid composition therein, and a corresponding activation unit.

FIG. 1D schematically depicts an embodiment of a device for plasma activating a liquid medium maintained in a can.

FIG. 1E schematically depicts an embodiment of an inlet tube and a hollow needle employed as an electrode.

FIG. 2A depicts another embodiment of a device for plasma activating a liquid medium in a can.

FIG. 2B schematically depicts yet another embodiment of a device for plasma activating a liquid medium in a can.

FIG. 3A illustrates schematically an embodiment of a sealed can comprising a liquid medium according to the teachings herein.

FIG. 3B illustrates schematically an embodiment of a device for plasma activating a liquid medium contained in the can of FIG. 3A.

FIG. 4A illustrates an embodiment of a passive chiller comprising an ice-pack in a form of a torus, surrounding a can.

FIG. 4B is an exploded view of an embodiment of a passive chiller comprising an ice-pack having an external chamber and an internal chamber encompassed by the external chamber.

FIG. 4C illustrates an embodiment of a device for plasma activating a liquid medium, the device incorporating a passive chiller of the invention.

FIG. 5 shows an image of agar plates onto which secretions derived from an infected ear were incubated and treated with a plasma-activated composition (left) or control (right).

FIG. 6 schematically depicts an embodiment of an endoscope employed to deliver a plasma-activated composition in a liquid form, into a cyst in a pancreas of a patient.

### DETAILED DESCRIPTION

The present invention provides compositions containing RONS useful in treating various diseases and disorders. The compositions are advantageously administered in a liquid form and are capable of undergoing a phase transition to a gel form in response to a stimulus, e.g., when in contact with the site of action. In this manner, increased residence time at the site of action is afforded thereby improving therapeutic efficacy.

Disclosed herein for the first time are compositions comprising RONS generated by plasma, wherein the compositions gel in situ, i.e., at the site of action, to afford the controlled release of RONS for prolonged, long or extended durations. The compositions are highly effective in treating pre-malignant conditions, malignant tumors, and preventing recurrence of malignancies following surgical excisions. The compositions are also useful in treating viral, bacterial, yeast, mold, or fungal infections, particularly of the ear or skin.

According to the principles of the present invention, the liquid compositions disclosed herein undergo an in-situ phase transition to a gel form in response to a stimulus. The term “phase transition” as used herein refers to a liquid-gel phase transition meant to encompass an increase in the viscosity or stiffness of the composition so as to provide longer residence time at the site of action. It is to be understood that the transition may be reversible or irreversible, with each possibility representing a separate embodiment.

Thus, provided herein is a composition that has been activated by plasma thereby comprising a plurality of reactive oxygen and nitrogen species. According to the principles of the present invention the composition includes a plurality of charged ions, radicals and electrons, with a net charge which is substantially neutral. The term “reactive oxygen and nitrogen species” or “RONS” as used herein refers to reactive moieties that contain oxygen, nitrogen or both. Typically, the reactive moieties are characterized by a short half-life. Included within this term are moieties such as, but not limited to, H₂O₂ (hydrogen peroxide), OH* (hydroxyl radical), HO₂* (hydroperoxy radical), O₂⁻ (superoxide radical), O₃ (ozone), NO* (nitric oxide), NO₂⁻ (nitrogen dioxide), NO₃⁻ (nitrate), and ONOO* (peroxynitrite). Each possibility represents a separate embodiment.

According to the teachings herein, plasma is preferably (but not necessarily) generated in the arcing mode, at ambient pressure. Arcing mode is characterized by volatile filamentous discharge, and was found by the inventors to be highly effective in generating reactive species as is described herein. Typical rates of generation of the reactive species H₂O₂, NO₂⁻ and NO₃⁻ by this method were found to be 10 mg/l, 60 mg/l and 300 mg/l, respectively, in 5 cc of a liquid composition of the invention, in 2.5 minutes. Typically, the amount of RONS in the composition ranges from about 1 mg/L to about 2,000 mg/L, including each value within the specified range. Exemplary ranges include, but are not limited to, about 10 mg/L to about 1,500 mg/L, about 50 mg/L to about 1,200 mg/L, or about 100 mg/L to about 1,000 mg/L, including each value within the specified ranges. Each possibility represents a separate embodiment.

A liquid, particularly a liquid composition comprising a polymer, configured to transform to a gel when exposed to a stimulus, may be plasma activated by various methods. Any method capable of plasma-activating a liquid is contemplated in the context of the current invention. Plasma activation is meant to include plasma generation within the considered medium (namely within the liquid) and—additionally or alternatively—in a gaseous medium which is being in contact or brought to contact with the medium. Further, to be considered ‘plasma activated’ in the context of the present invention, the medium must include, as a result of the process of plasma activation as described above, excited species—e.g., ROS and RNS—that are not present in the medium in the absence of activation. Explicit examples for plasma activation of a liquid by exposing the liquid to a plasma-excited gas, are provided in the examples herein below, particularly EXAMPLE 2 to EXAMPLE 7.

According to the principles of the present invention, the composition comprises a biocompatible polymer which is capable of gelation in response to a stimulus. Thus, the composition is configured to undergo a phase transition to a gel form upon a stimulus. In some aspects and embodiments, the phase transition is reversible.

In certain aspects and embodiments, the phase transition is induced by a change in temperature. In some embodiments the phase transition may be induced by a decrease in temperature. Thus, for example, a composition in a liquid form may be plasma activated at a temperature higher than body temperature. The composition may then transform to gel upon applying the activated composition to a body part and upon reaching the body temperature or even below body temperature.

However, phase transitions from a liquid form to gel are often gradual (as a function of temperature). This is to say that a significant temperature difference—e.g., 10 or 15 degrees or even 20 degrees—may exist between the gel phase and the liquid phase; hence, to ensure low viscosity during the plasma treatment, the composition must be at a temperature significantly higher than the body temperature which ensures gelation. Thus, a possible disadvantage of this approach is that applying to a subject the composition at a temperature in which it is liquid, namely significantly higher than body temperature, may cause unpleasantness, pain or even actual damage.

In some embodiments, the phase transition may be induced by an increase in temperature. For example, the phase transition may be induced when reaching a temperature of 15° C. or higher. In other embodiments, the phase transition is induced when reaching a temperature of 20° C. or higher. In further embodiments, the phase transition is induced when reaching a temperature of 25° C. or higher. Currently preferred is the phase transition that occurs upon contacting a tissue at physiological body temperatures. Thus, within the scope of the present invention are compositions comprising thermoreversible gel-forming polymers that are liquid at low temperatures and are capable of undergoing a phase transition to a gel form at body temperatures where they are designed to exert their therapeutic effect thereby allowing the slow release of the RONS at a site of choice.

According to some embodiments, the phase transition may be induced by a change of acidity (pH). In some embodiments, a liquid composition of the invention may gel following mixing with an aqueous solution such that its acidity (or basicity) on the pH scale changes by more than 1.

According to some embodiments, a liquid composition of the invention may gel by light-induced cross-linking, as is further detailed below.

According to the principles of the present invention, the polymers used in the compositions are biocompatible polymers. In some aspects and embodiments, the polymers are biodegradable.

Within the scope of the present invention are synthetic polymers as well as natural polymers with each possibility representing a separate embodiment. Suitable polymers include, but are not limited to, polyanhydrides; poly(sebacic acid) SA; poly(ricinoleic acid) RA; poly(fumaric acid), FA; poly(fatty acid dimmer), FAD; poly(terephthalic acid), TA; poly(isophthalic acid), IPA; poly(p-{carboxyphenoxy}methane), CPM; poly(p-{carboxyphenoxy}propane), CPP; poly(p-{carboxyphenoxy}hexane) CPH; polyamines, polyurethanes, polyesteramides, polyorthoesters {CHDM: cis/trans-cyclohexyl dimethanol, HD:1,6-hexanediol, DETOU: (3,9-diethylidene-2,4,8,10-tetraoxaspiro undecane)}; polydioxanones; polyhydroxybutyrates; polyalkylene oxalates; polyamides; polyesteramides; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic cellulose esters; polyacrylic acids; polybutyric acid; triblock copolymers (PLGA-PEG-PLGA), triblock copolymers (PEG-PLGA-PEG), poly (N-isopropylacrylamide) (PNIPAAm), poly (ethylene oxide)-poly (propylene oxide)-poly (ethylene oxide) tri-block copolymers (PEO-PPO-PEO), poly valeric acid; polyethylene glycol; polyhydroxyalkylcellulose; chitin; chitosan; polyorthoesters and copolymers, terpolymers; lipids such as cholesterol, lecithin; poly(glutamic acid-co-ethyl glutamate), poly (D,L-lactide-co-glycolide) (PLGA), poly (D,L-lactide) (PLA), polyglycolide (PGA), polycaprolactone (PCL), polyhydroxybutyrate, polyorthoesters, polyalkaneanhydrides, gelatin, alginate, collagen, oxidized cellulose, polyphosphazene, and any combination thereof. Each possibility represents a separate embodiment.

Currently preferred polymers according to the principles of the present invention are synthetic biocompatible polymers. Without being bound by any theory or mechanism of action, it is contemplated that the use of a synthetic polymer provides a means of creating biocompatible hydrogels having controlled physical properties such as density, stiffness, and proteolytic degradability, through the versatile synthetic components, without compromising biocompatibility. Additional possible advantage to the use of the synthetic biocompatible polymers within the scope of the present invention may be the reversibility of the phase transition that avoids the use of crosslinking agents and is controlled by stimuli present in physiological conditions. In some embodiments, the polymer used in the compositions disclosed herein is not a natural polymer such as alginate or gelatin.

Within the scope of the present invention are synthetic biocompatible polymers comprising polyoxyethylene-polyoxypropylene block copolymers known as “poloxamers”. Exemplary and non-limiting poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, or a mixture thereof. Each possibility represents a separate embodiment. Currently preferred are poloxamer 407, poloxamer 188, and poloxamer 338, or a mixture thereof. Each possibility represents a separate embodiment.

In particular aspects and embodiments, the composition comprises a mixture of poloxamer 407 and poloxamer 188. Suitable weight ratios of poloxamer 407 and poloxamer 188 include, but are not limited to, ratios of 10:1 to 1:1, including any ratio therebetween. Currently preferred are ratios of 6:1 to 1:1, for example 4:1.

In specific aspects and embodiments, the composition comprises a mixture of poloxamer 338 and poloxamer 188. Suitable weight ratios of poloxamer 338 and poloxamer 188 include, but are not limited to ratios of 10:1 to 1:1, including any ratio therebetween. Currently preferred are ratios of 6:1 to 1:1, for example 4:1.

In various aspects and embodiments, the phase transition is induced by a change in pH, for example when reaching an acidic pH or a neutral to basic pH. Each possibility represents a separate embodiment. In order to achieve a phase transition in response to a pH, the composition typically contains a pH-responsive polymer for example an enteric polymer or a reverse-enteric polymer. Each possibility represents a separate embodiment.

The term “enteric polymer” as used herein refers to a polymer that is characterized by increase in permeability at pH values of above pH 5.0 (e.g., intestinal fluid) while remaining insoluble at low pH values, such as those found in the environment of the stomach. Exemplary and non-limiting enteric polymers include acrylic and (meth)acrylate acid copolymers, polyvinyl acetate phthalate, and the like. Each possibility represents a separate embodiment. Acrylic and methacrylate acid copolymers are anionic copolymers based on (meth)acrylic acid and alkyl (meth)acrylate, such as, but not limited to, polymethacrylic acid, polymethyl methacrylate, polyethyl methacrylate, and polyethyl acrylate among others. Commercial acrylic and methacrylate acid copolymers are available under the trade name Eudragit (Evonik Industries AG, Essen, Germany) and are typically provided as powder or aqueous dispersions, including, but not limited to, Eudragit L 30 D-55, Eudragit L 100-55, Eudragit L 100, Eudragit L 12.5, Eudragit NE 40 D, Eudragit RL 100, Eudragit S 100, Eudragit S 12.5, Eudragit FS 30 D, Eudragit RL PO, Eudragit RL 12.5, Eudragit RL 30 D, Eudragit RS 100, Eudragit RS PO, Eudragit RS 30 D, Eudragit RS 12.5, Eudragit NE 30 D, Eudragit NM 30 D, or combinations and mixtures thereof. Each possibility represents a separate embodiment.

A “reverse enteric polymer” as used herein refers to polymers which are insoluble at pH values greater than those found in the stomach i.e., at pH values greater than 5.0 while being soluble at acidic pH values. Exemplary reverse enteric polymers include, but are not limited to, a (meth)acrylate polymer or copolymer, such as acrylate and methacrylate copolymers having primary, secondary or tertiary amino groups or quaternary ammonium groups. These reverse enteric polymers are commercially available as Eudragit E 100, Eudragit E 12.5, Eudragit EPO, Eudragit RL 100, or combinations and mixtures thereof. Each possibility represents a separate embodiment.

Typically, the amount of synthetic biocompatible polymer in the composition ranges from about 1% to about 50% of the total weight of the composition, including each value within the specified range. Currently preferred amounts include those above 5% of the total weight of the composition. Exemplary amounts include, but are not limited to, about 1%, about 5%, about 10%, about 15%, about 20%, about 25%, about 30%, about 35%, about 40%, about 45%, or about 50% of the total weight of the composition, with each possibility representing a separate embodiment.

According to various aspects and embodiments, the synthetic biocompatible polymer is dispersed or dissolved in a fluid medium which may further comprise a buffering or a pH adjusting agent and a solvent. According to the principles of the present invention one or more buffering agents or pH adjusting agents may be included in the composition. Such buffering or pH adjusting agents include, but are not limited to, 2-amino-2-hydroxymethyl-1,3-propanediol (Tris), 2-[bis(2-hydroxyethyl)imino]-2-(hydroxymethyl)-1,3-propanediol (bis-Tris), 4-morpholine ethane sulfonic acid (MES) buffer, ammonium chloride, bicine, tricine, sodium phosphate monobasic, sodium phosphate dibasic, sodium carbonate, sodium bicarbonate, sodium acetate, sodium phosphate, glutamic acid, citrate buffer, histidine buffer, Dulbecco's phosphate-buffered saline, 4-(2-hydroxyethyl)-1-piperazineethanesulfonic acid (HEPES), methoxypsoralen (MOPS), N-cyclohexyl-3-aminopropanesulfonic acid (CAPS), N-cyclohexyl-2-hydroxyl-3-aminopropanesulfonic acid (CAPSO), N-Cyclohexyl-2-aminoethanesulfonic acid (CHES), 3-[4-(2-Hydroxyethyl)-1-piperazinyl]propanesulfonic acid (HEPPS), phosphate-buffered saline, tris-buffered saline, Hank's solution, and Ringer's solution or a mixture or combination thereof. Each possibility represents a separate embodiment.

According to various aspects and embodiments, the composition has a pH in the range of about 5.0 to about 7.5, including each value within the specified range. Exemplary non-limiting ranges include about 5.3 to about 6.3, about 5.8 to about 6.8, about 6.0 to about 7.0 etc., including each value within the specified ranges. For example, the composition of the present invention may have a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4 or about 7.5, with each possibility representing a separate embodiment.

In various aspects and embodiments, the fluid medium in which the polymers are dispersed or dissolved is an aqueous medium comprising water or saline as solvents. Typically, the amount of solvent ranges from about 50% to about 99% of the total weight of the composition, including each value within the specified range. Exemplary amounts include, but are not limited to, about 50%, about 55%, about 60%, about 65%, about 70%, about 75%, about 80%, about 85%, about 90%, about 95%, or about 99% of the total weight of the composition, with each possibility representing a separate embodiment.

In some aspects and embodiments, the composition further comprises an ionic tonicity agent, such as sodium chloride or a non-ionic tonicity agent such as a polyol. Additional excipients that may be present in the composition include, but are not limited to surfactants and preservatives as is known in the art.

According to the principles of the present invention, the compositions disclosed herein form a gel (a hydrogel) upon a stimulus such as a change in temperature or pH or light-induced cross linking. The term “hydrogel” according to the present invention refers to a three-dimensional hydrated assembly of biocompatible nanofibers. A liquid-gel phase transformation may be characterized by an increase of the material's viscosity, wherein the increase is by at least one order of magnitude over a range of 20 degC. or less. Typically, the gel is characterized by viscosities greater than 20,000 mPa·s, preferably greater than 50,000 mPa·s and even more preferably greater than 100,000 mPa·s. Each possibility represents a separate embodiment. In the liquid phase, the composition is typically characterized by viscosities lower than 600 mPa·s, preferably lower than 300 mPa·s, and even more preferably lower than 100 mPa·s. Each possibility represents a separate embodiment. Measurements of viscosities can be performed as is known in the art, for example using a suitable viscometer including, but not limited to, a Brookfield Viscometer or an Anton Paar RheoPlus viscometer with an appropriate setup. It is commented that by determining the viscosity of the gel phase, the temporal profile of RONS release rate may be determined, whereby the greater the viscosity, the slower the release is.

In certain aspects and embodiments, the compositions disclosed herein are useful in the prophylaxis, treatment, and post-treatment of cancer. The term “cancer” as used herein refers to a disorder in which a population of cells has become, in varying degrees, unresponsive to the control mechanisms that normally govern proliferation and differentiation. Cancer refers to various types of malignant neoplasms and tumors, including primary tumors, and tumor metastasis. Non-limiting examples of cancers which can be treated by the compositions of the present invention are brain, ovarian, colon, prostate, kidney, bladder, breast, lung, oral, and skin cancers. Each possibility represents a separate embodiment. In one embodiment, the cancer is a soft-tissue cancer. Specific examples of cancers include, but are not limited to, carcinomas, sarcomas, myelomas, leukemias, lymphomas and mixed type tumors. Each possibility represents a separate embodiment. Additional examples of cancer include, but are not limited to, lymphoproliferative disorders, breast cancer, ovarian cancer, prostate cancer, cervical cancer, endometrial cancer, liver cancer, bladder cancer, stomach cancer, colon cancer, colorectal cancer, pancreatic cancer, cancer of the thyroid, esophagus cancer, head and neck cancer, cancer of the central nervous system, cancer of the peripheral nervous system, skin cancer, kidney cancer, as well as metastases of all the above. Each possibility represents a separate embodiment. Particular types of cancers include, but are not limited to, hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, leukemia, multiple myeloma, myeloid lymphoma, Hodgkin's lymphoma, non-Hodgkin's lymphoma, and hepatocarcinoma. Each possibility represents a separate embodiment.

In some representative embodiments, the cancer is selected from the group consisting of colorectal cancer and bladder cancer. Each possibility represents a separate embodiment.

The term “treatment of cancer” in the context of the present invention includes at least one of the following: a decrease in the rate of growth of the cancer (i.e., the cancer still grows but at a slower rate); cessation of growth of the cancerous growth, i.e., stasis of the tumor growth, and, in preferred cases, the tumor diminishes or is reduced in size. The term also includes reduction in the number of metastases, reduction in the number of new metastases formed, slowing of the progression of cancer from one stage to the other and a decrease in the angiogenesis induced by the cancer. In most preferred cases, the tumor is totally eliminated. Additionally included in this term is lengthening of the survival period of the subject undergoing treatment, lengthening the time of diseases progression, tumor regression, and the like. It is to be understood that the term “treating cancer” also refers to the inhibition of a malignant (cancer) cell proliferation including tumor formation, primary tumors, tumor progression or tumor metastasis. The term “inhibition of proliferation” in relation to cancer cells, may further refer to a decrease in at least one of the following: number of cells (due to cell death which may be necrotic, apoptotic or any other type of cell death or combinations thereof) as compared to control; decrease in growth rates of cells, i.e. the total number of cells may increase but at a lower level or at a lower rate than the increase in control; decrease in the invasiveness of cells (as determined for example by soft agar assay) as compared to control even if their total number has not changed; progression from a less differentiated cell type to a more differentiated cell type; a deceleration in the neoplastic transformation; or alternatively the slowing of the progression of the cancer cells from one stage to the next. Each possibility represents a separate embodiment.

A particular population to which the compositions of the invention is beneficial includes subjects that have pre-malignancies. Thus, there is provided the use of a composition as disclosed herein for the preparation of a medicament for treating a pre-malignant lesion. As used herein, the term “pre-malignant” refers to a cyst, a polyp, a lesion and the like or any form of cell structural or growth disorder which is not malignant but has an increased probability of becoming cancerous. In particular, this term refers to a tissue that exerts one or more of: morphologically or architectural changes, histological alterations showing atypia of cells or dysplasia, and initial molecular changes in gene expression or expression of specific isoforms of proteins. Each possibility represents a separate embodiment. Particular types of pre-malignancies include, but are not limited to, myelodysplastic disorders, cervical carcinoma-in-situ, familial intestinal polyposes (e.g., Gardner syndrome), oral leukoplakias, histiocytosis, keloids, hemangiomas, hyperkeratosis, and papulosquamous eruptions. Each possibility represents a separate embodiment. Also included are non-cancerous hyperproliferative diseases such as warts and psoriasis. Each possibility represents a separate embodiment.

An additional patient population suitable for being treated with the compositions disclosed herein are subjects that underwent tumor excision in order to prevent or delay tumor recurrence. Thus, there is provided the use of a composition as disclosed herein for the preparation of a medicament for preventing or delaying tumor recurrence following tumor excision. Cancer recurrence after excision is a known problem in many cancer types typically attributed to the failure to remove all cancer cells during surgery, spread of cancer cells during percutaneous ablation, and resistance to chemotherapeutic agents. The compositions disclosed herein are particularly beneficial in preventing or delaying tumor recurrence following tumor excision due to their increased residence time at the site of application.

Within the scope of the present invention is a liquid composition comprising a plurality of reactive oxygen and nitrogen species generated by plasma for use in preventing or delaying tumor recurrence following tumor excision. Thus, in accordance with these embodiments, there is provided a method of preventing or delaying tumor recurrence following tumor excision in a subject in need thereof, the method comprising the step of contacting the remaining tissue that surrounded the tumor with a therapeutically effective amount of a liquid composition comprising a plurality of reactive oxygen and nitrogen species generated by plasma. In one embodiment, the subject in need thereof is afflicted with bladder cancer.

As used herein, the term “contacting” in the therapeutic context set forth above refers to topical administration, i.e., bringing in contact with the compositions of the present invention. Contacting can be accomplished to cells or tissue cultures, or to living organisms, for example humans. Thus, the term “contacting” may be ex-vivo on a surface, on a device, in cell/tissue culture dish etc., or in-vivo e.g., inside a living organism.

Encompassed by the present invention is the treatment of infections such as bacterial, viral, yeast, mold, and/or fungal infections using the compositions disclosed herein. The term “treating” as used herein with reference to infections denotes stopping or slowing down the progression of the disease. The term “treating” further includes the reduction in the occurrence of various symptoms associated with the infection. In one embodiment, with reference to bacterial or viral infections, treating comprises the inhibition of bacterial or viral replication accompanied by the reduction of bacterial or viral load. In other embodiments, treatment comprises essentially complete eradication of the infectious disease.

Particular bacteria types include, but are not limited to, gram positive bacteria and gram-negative bacteria. Exemplary bacteria include species of Streptococcus, Staphylococcus, Haemophilus, Moraxella, Micrococcus, Corynebacterium, Clostridium, Pseudomonas, Proteus, Peptostreptococcus, Neisseria, and Escherichia. Each possibility represents a separate embodiment.

Viral infections which are treated, inhibited, attenuated or suppressed by the compositions disclosed herein include, but are not limited to, herpesvirus infections such as, herpes simplex virus (HSV) type 1, HSV type 2, varicella-zoster virus, cytomegalovirus, Epstein-Barr virus (EBV), human herpesvirus 6 (variants A and/or B), human herpesvirus 7, and human herpesvirus 8 (Kaposi's Sarcoma associated Herpes Virus); Human Papilloma Virus (HPV); Molluscum Contagiosum Virus (WV) as well as skin diseases associated with viral infections such as warts and benign tumors of the skin and/or mucosa which are caused by papilloma viruses, for example Verrucae plantares, Verrucae vulgares, Verrucae planae juveniles, Epidermodysplasia verruciformis, Condylomata acuminata, Condylomata plana, Bowenoid papulosis, Papillomas on the larynx and oral mucosa, and focal epithelial hyperplasia. Each possibility represents a separate embodiment.

Within the scope of the present invention is the treatment of fungal infections which include infections caused a fungus or yeast. The infection may occur in the skin, fingernails, toenails, and mucosal membranes including, but not limited to, mouth, pharynx, esophagus, lung, and genitalia (including vagina and penis), or may be systemic, for example in immunocompromised patients. Each possibility represents a separate embodiment.

Pathogenic yeasts include, but are not limited to, various species of the genus Candida, for example, Candida auris, Candida albicans, Candida glabrata, Candida enolase, Candida tropicalis, Candida krusei, Candida parapsilosis, Candida stellatoidea, Candida parakawsei, Candida lusitaniae, Candida pseudotropicalis, Candida viswanathii, and Candida guilliermondii, as well as species of Aspergillus, Rhizopus, Mucor, Histoplasma, Coccidioides, Blastomyces, Trichophyton, Microsporum, and Epidermophyton such as, but not limited to Aspergillus fumigatus, Aspergillus flavus, Aspergillus niger, Histoplasma capsulatum, Coccidioides immitis, and Blastomyces dermatitidis. Each possibility represents a separate embodiment.

Encompassed by the present invention are infections to the skin, ear, eye, nose, throat, lungs, gastro-intestinal tract, urinary tract, or genitalia. Each possibility represents a separate embodiment. In some representative embodiments, the infection is an ear infection known as Otitis. In other representative embodiments, the infection is a skin infection, for example Propionibacteriium acne resulting in juvenile acne.

The compositions disclosed herein are administered in therapeutically effective amounts. A “therapeutic” treatment is a treatment administered to a subject who exhibits signs of pathology for the purpose of diminishing or eliminating those signs. A “therapeutically effective amount” is that amount of the moieties generated by plasma which is sufficient to provide a beneficial effect to the subject to which the composition is administered. Typically, the subject is a mammal, e.g., a human, a pet or any domesticated animal. In some aspects and embodiments, the compositions disclosed herein are intended for veterinary use.

Determining the therapeutically effective amount is well within the capability of those skilled in the art, especially in light of the disclosure provided herein. The exact amount of RONS can be chosen by the individual physician in view of the patient's condition. It typically depends on certain parameters of the subject being treated, for example, weight, age, and the severity of the disease. The composition may be administered as a single dose or multiple doses in a continuous or intermittent manner. The term “intermittent” as used herein refers to stopping and starting at either regular or irregular intervals. For example, intermittent administration can be administration every day for a certain period of time or administration in cycles or administration on alternate days. Each possibility represents a separate embodiment.

The compositions of the present invention are typically activated by plasma prior to being administered or brought into contact with the site of therapeutic use. Explicit examples of methods of plasma activation of the compositions of the invention are provided in the examples below.

According to some embodiments, the compositions according to the present invention may include pharmaceutical agents such as antibiotics and/or steroids. Such pharmaceutical agents may be added to the composition of the invention during processing, plausibly after plasma excitation and before applying the composition to a patient.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to ±10% of the reference value, or the larger of the above and ±1 degree Celsius if the value refers to degrees Celsius (denoted herein degC).

It is noted that as used herein and in the appended claims, the singular forms “a”, “an”, and “the” include plural referents unless the context clearly dictates otherwise. Thus, for example, reference to “a synthetic biocompatible polymer” includes a plurality of such polymers and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a composition having at least one of A, B, and C” would include but not be limited to compositions that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples. Various embodiments and aspects of the present invention as delineated hereinabove and as claimed in the claims section below find experimental support in the following examples.

### EXAMPLES

### Example 1

### Compositions

Compositions comprising a mixture of poloxamers were prepared while constant cooling and stirring (Table 1). First cold water (5° C.) was poured into a container, then while stirring, the poloxamers were slowly added. The compositions were homogenized by constant stirring for 2 hours.

TABLE 1

| | Mass % | gram |
|---|---|---|
| Poloxamer 407 | 20 | 4.0 |
| Poloxamer 188 | 5 | 1.0 |
| Water | 75 | 15.0 |
| Total | 100 | 20.0 |

Additional compositions as outlined in Table 2 are prepared in the same manner.

TABLE 2

| | Mass | | Mass | | Mass | | Mass | |
|---|---|---|---|---|---|---|---|---|
| | % | gr | % | gr | % | gr | % | gr |
| Poloxamer | 20 | 4.0 | 15 | 3.0 | | | | |
| 407 | | | | | | | | |
| Poloxamer | | | | | 20 | 4.0 | 15 | 3.0 |
| 338 | | | | | | | | |
| Poloxamer | 5 | 1.0 | 5 | 1.0 | 5 | 1.0 | 5 | 1.0 |
| 188 | | | | | | | | |
| Buffer 6.5 | 75 | 15.0 | 80 | 16.0 | 75 | 15.0 | 80 | 16.0 |
| 0.05M | | | | | | | | |
| Total | 100 | 20.0 | 100 | 20.0 | 100 | 20.0 | 100 | 20.0 |

### Example 2

### Treatment of Ear Infection in Pets

Otitis externa is an inflammation of the external ear canal distal to the tympanic membrane. It is one of the most common reasons for pets—especially dogs—to be presented to the veterinarian. The inflammation may be provoked by one of several primary causes such as foreign bodies in the canal, allergy, parasites, autoimmune disorder and others. However, prolonged irritation by a primary cause might evolve to infection caused by fungus, yeast (e.g., Malassezia) or bacteria (Staphylococcus, Streptococcus, Enterococcus, Pseudomonas, Proteus, etc.). Lack of treatment might lead to increased irritation, increased inflammation, pain, progression of the inflammation into the middle ear and damage, up to loss of hearing. The most common method of treatment includes application of antibiotics and anti-fungal medications to disinfect the ear. However, the growing reluctance of using antibiotics and the resulting tendency to adopt alternative methods of treatment, promotes a need for plasma-based disinfecting treatments.

Thus, according to some embodiments, a plasma-activated medium (PAM) may be applied to a subject's ear—in particular a pet's ear—to disinfect the ear and decrease or eliminate inflammation. In some embodiments, a liquid composition comprising a synthetic biocompatible polymer capable of in-situ gelation may be prepared as described above. A preferred stimulus of gelation may be selected to be elevation of temperature from typical room temperature (e.g., about 26° C.) to typical body temperature (e.g., about 37° C.). In some embodiments, the liquid composition may be transformed into gel upon heating to above 30° C. or above 35° C. The liquid composition may be activated by plasma as described in detail herein below. The resulting PAM may then be applied to the subject's ear, where the composition transforms into gel and stabilizes in situ. The plasma activated gel may thus release RONS onto the infected region for a pre-defined duration of several hours or several days following which the gel decomposes.

Activation of the liquid composition of the invention by plasma may be carried out in mass quantities at the factory or, additionally or alternatively, in small quantities, at the clinic, prior to application of the activated medium. Activation in the factory has an advantage of simplicity of use for the end customer, as the practitioner at the clinic is supplied with an activated liquid that may be readily applied. Additional advantages may be standardization of the product and possibly lower cost due to processing in large quantities. A disadvantage of plasma activation at the factory is the decrease of RONS concentration over time, which may lead to decreased efficacy. Thus, plasma activation of the composition at the clinic prior to application, may be preferred in some embodiments.

Regardless of whether plasma activation is carried out in mass quantities or in small quantities, at the factory or at the clinic prior to application, the temperature of the activated medium should preferably be kept well below the gelation temperature during activation.

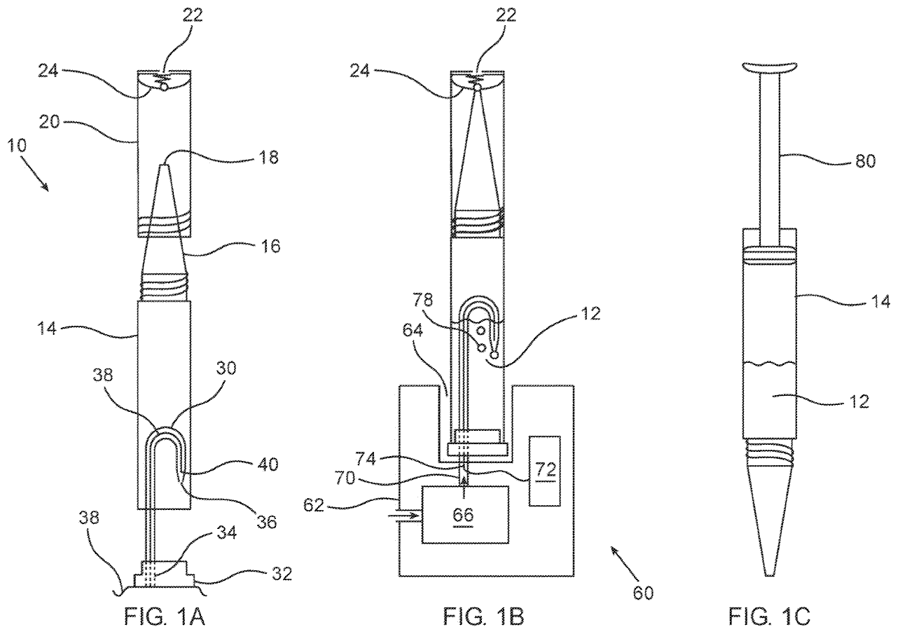

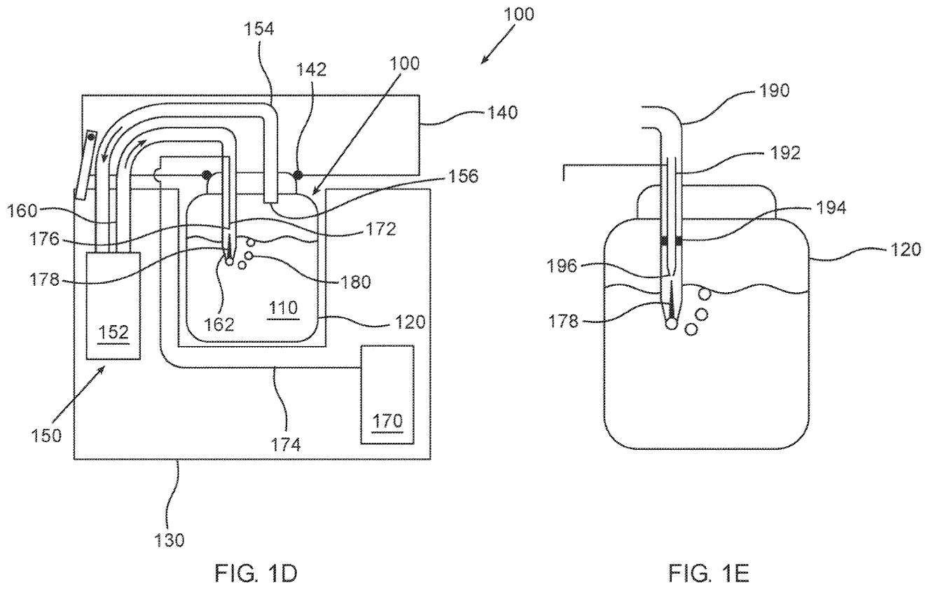

FIGS. 1A-1C depict schematically an embodiment of a capsule 10 (FIG. 1A) configured to contain a liquid composition comprising a polymer 12 therein (FIG. 1B). The liquid composition may be transformed into gel upon heating, e.g., upon heating from a typical room temperature to a typical body temperature. In some embodiments, the liquid composition may be transformed into gel upon heating to above 30° C. or above 35° C. A corresponding activation unit 60 (FIG. 1B), may be employed for plasma activating the liquid composition inside the capsule. After plasma activation, the capsule may be employed as a syringe barrel 14 for applying the activated liquid using a plunger 80 (FIG. 1C).

FIG. 1A schematically depicts a semi-exploded view of an embodiment of capsule 10. Capsule 10 comprises syringe barrel 14, having a funnel 16 with an opening 18 at the distal end thereof, the funnel being configured to facilitate applying the liquid composition inside the subject's ear after plasma activation. Syringe barrel 14 may be made of a dielectric material such as glass or plastic. In some embodiments syringe barrel 14 may be made of metal. Capsule 10 further comprises a funnel cup 20 detachable from the syringe barrel 14 and configured to cover the funnel and thereby to seal funnel opening 18 against leakage of the liquid composition outwards and against penetration of contamination into the syringe barrel. Funnel cap 20 comprises a cap opening 22 at the distal end thereof, and a one-way valve 24 configured to allow pressurized gas to flow out from the syringe barrel and from funnel opening 22 to the ambient during plasma activation, as is further explained below.

Capsule 10 further comprises a bent tube 30 passing through a cork 32 and extending between a tube port 34 at the cork and a tube distal end 36. Bent tube 30 may be composed of a dielectric material such as glass or plastic. A needle electrode 38 is arranged concentrically along the bent tube, having a pointed tip 40 near tube distal end 36 and, in some embodiments, retracted from the tube distal end towards the inside of the tube.

During manufacturing and assembly of capsule 10, syringe barrel 14 may be filled with liquid composition comprising the polymer 12 and then sealed in both ends by funnel cap 20 and by cork 32, respectively. Tube port 34 may be further sealed by a detachable seal 38. Detachable seal 38 may be a detachable cover, e.g., a sticky foil, configured to seal tube port 34 and prevent penetration of contamination into tube 30. It is noted that the amount of the liquid composition inside syringe barrel 14 may be calibrated so that, when capsule 10 is positioned with funnel 16 pointing upwards, as depicted in FIG. 1B, the liquid surface is below the bend of bent tube 30 and above tube distal end 36.

Plasma activation of liquid composition 12 may be carried out using activation unit 60, as is depicted schematically in FIG. 1B. Activation unit 60 comprises a housing 62 having a slot 64 dimensioned and configured to receive capsule 10 therein. Activation unit 60 further comprises a compressor 66 configured to compress air from the ambient towards a slot port 70. Additionally or alternatively, in some embodiments, a gas other than air may be used. In some embodiments a pressurized gas source (not shown here) may be used together with compressor 66 or instead of compressor 66, to force the gas towards slot port 70. Such pressurized gas source may be a gas reservoir containing for example an inert gas such as nitrogen (N2), argon or helium, to name a few examples. Thus, in some embodiments, a plasma of some inert gas may be generated in the capsule 10, and in some embodiments a plasma of a mixture of air and another gas may be generated.

Activation unit 60 further comprises a HV power source 72 configured to generate high voltage sufficient to apply a plasma generating electric field. HV power source may generate a voltage typically above 10 KV or above 6 KV or above 3 KV or even above 1 KV. Each possibility represents a separate embodiment. The HV supplied by HV power source 72 may be constant (DC) or alternating (AC). In some embodiments, the HV may be at radio frequency (RF), between 3 KHz and 10 GHz. HV power source 72 is electrically associated with a HV connector 74 in slot 64. In some embodiments, the HV connector is part of the slot port 70. It is noted that the term “electromagnetic field” (or EM field) may thus include also electrostatic field.

For plasma activation of the liquid composition, capsule 10 with liquid composition 12 inside, is placed in slot 64 of activation unit 60 as is depicted in FIG. 1B. Tube port 34 connects to slot port 70 so that compressor 66 is in flow communication with bent tube 30 and HV power source 72 is electrically associated with electrode 38 via HV connector 74. In some embodiments, where syringe barrel 14 is metallic, it may be connected to ground potential. When HV power source 72 is activated, a plasma generating electric field is applied between electrode pointed tip 40 and the liquid composition, the liquid being in ground potential, thereby generating plasma in that space.

Because the plasma is generated at ambient pressure, by an electric field applied between a pointed electrode (pointed tip 40 in the current embodiment) and the liquid, the plasma is generated in arcing mode. Arcing mode is characterized by volatile filamentous discharge, and was found by the inventors to be highly effective in generating reactive species as is described herein.

Typically, compressor 66 is activated together with HV power source so that air from the ambient is compressed into bent tube 30 to be released via bubbles 78 and via one way valve 24 to the ambient again. The generation of the bubbles in liquid composition 12 wherein the bubbles are filled with excited air assists in increasing the liquid surface that is affected by the plasma as well as agitating and mixing the liquid composition, thereby assisting in preventing heating of a limited portion of the liquid composition, and thus preventing undesired gelation due to local temperature rise in the liquid.

According to some embodiments, funnel cap 20 is only used for tightly sealing funnel opening 18 and does not comprise a one-way valve. In such embodiments, funnel cap 20 may be removed from the capsule after the capsule is placed in the slot 64 but prior to plasma activation, and the air escaping from the bent tube through the bubbles is freely released to the ambient via funnel opening 18.

After plasma activation is complete, capsule 10 may be removed from slot 64 and taken for application of the liquid composition in a subject's ear. While holding the capsule with barrel funnel 16 pointing downwards, cork 32 may be extricated from barrel 14 and plunger 80 may be slightly inserted there instead. Funnel cap 20 may then be removed, as is depicted in FIG. 1C. In this configuration, the syringe comprising the barrel 14 with the plunger 80 may be taken for application of the liquid composition in the subject's ear.

### Example 3

### Plasma Activation of a Liquid Medium

FIG. 1D schematically depicts an embodiment of a device 100 for plasma activating a liquid medium 110, the liquid medium being stored in a can 120. In some embodiments it may be undesired to allow the temperature of the liquid medium to rise above a given temperature during plasma activation. In some embodiments it may be desired to maintain the temperature of the liquid medium below room temperature. It may thus be advantageous, in some embodiments, to apply cooling to the liquid medium, directly or indirectly.

In some embodiments the liquid medium 110 may be transformed into gel upon heating, e.g., upon heating from a typical room temperature to a typical body temperature. In some embodiments, the liquid medium may be transformed into gel upon heating to above 30° C. or above 35° C. Can 120 is configured for storing and possibly for transporting the liquid medium. Can 120 may be made of a dielectric material such as glass or plastic. In some embodiments, can 120 may be metallic. Can 120 may be closed with a cover (not shown here) which may be used for hermetically sealing the can, and should be removed prior to the plasma treatment described here. Additionally or alternatively, can 120 may be hermetically sealed (namely sealed against passage of gas therethrough) or biologically sealed (sealed against penetration of bacteria or viruses) using a foil seal (not shown here), which is removed by hand or torn prior to the plasma treatment.

Device 100 comprises a housing 130 having a slot 132 dimensioned and configured to receive can 120 therein, and a movable door 140 configured to close over the slot. Door 140 comprises a seal 142 configured to seal the opening of can 120 when can 120 is in slot 132 and door 140 is closed.

Device 100 further comprises a gas circulating system 150 configured to circulate gas through can 120 and through the liquid composition during plasma activation. Gas circulating system 150 comprises a compressor 152 configured to collect gas from an outlet pipe 154 and compress the gas into an inlet pipe 160. Outlet pipe 154 extends between compressor 152 and an outlet pipe distal end 156. Outlet pipe distal end 156 is open ended, and configured to come to flow communication with the top portion of can 120, thereby functioning as a gas outlet port of can 120, when the can is in slot 132 and the door 140 is closed. Likewise, inlet pipe 160 extends between compressor 152 and an inlet pipe distal end 162. Distal end 162 is open ended, and is configured to penetrate into can 120 and establish flow communication with the fluid maintained in the can, thereby functioning as a gas inlet port of can 120, when the can is in slot 132 and the door 140 is closed.

Device 100 further comprises a high voltage (HV) power source 170 electrically associated with an electrode 172 configured to apply a plasma generating EM field when supplied with a suitable HV. Electrode 172 may be, in some embodiments, a pointed electrode. In some embodiments, electrode 172 may be a needle as is described in the Figure. Electrode 172 may be electrically associated with HV power source 170 by a Galvanic contact via an electric cord 174, although other forms of electrical associations, e.g., capacitance coupling or inductive coupling, are contemplated. In some embodiments, where can 120 is metallic, the can may be electrically connected to ground potential, thereby attributing ground potential to the liquid medium 110 therein.

HV power source 170 may supply to electrode 172 HV constant in time (direct current—DC) or alternating (AC). In some embodiments, the HV is at a frequency greater than 3 KHz (termed RF). In some embodiments, the HV is at a frequency between 30 KHz and 10 GHz, including each value within the specified range. In some embodiments plasma 178 is generated in the arcing mode, between the electrode and the liquid medium. In some embodiments plasma is generated in the space above the liquid medium. In some embodiments the plasma-generating EM field is applied between electrode 172 and another component of device 100, e.g., the walls of can 120. In some embodiments plasma is generated between two electrodes (not shown here) neither of the two is pointed; for example between two electrodes shaped as sheets electrically isolated from each other and arranged opposing each other on the walls of can 120.

In some embodiments, the HV source may include a low-voltage to HV transformer (not shown here) for generating the HV supplied to electrode 172. In some embodiments, the HV transformer is a magnetic (inductance) step-up transformer as is known in the art. In some embodiments, the HV transformer is a piezoelectric HV transformer as is described for example in patent application publication No. WO 2021/144260. In some embodiments, the HV supplied to the electrode 172 is pulse-modulated (square-wave modulated) RF. Such pulse modulation is characterized by the duty cycle of the square wave namely by the ratio of the wave “on” time to the overall time period of the wave. Employing such modulated RF HV allows, by controlling the duty cycle of the modulation, relatively simple control over the power supplied by the HV power source to the electrode and hence over the average power of the generated plasma.

Electrode 172 preferably resides inside inlet pipe 160 having its pointed tip 176 near inlet pipe distal end 162. In some embodiments, pointed tip 176 is retreated towards the inside of the pipe by a small distance, e.g., between 0.1 mm-10 mm, including each value within the specified range.

During operation can 120 is positioned in slot 132 and the door 140 is closed, thereby sealing the interior of can 120 and preventing escape of gas from the can to the outside. Compressor 152 circulates air through circulating system 150 by compressing air into inlet pipe 160 and enforcing gas flow towards the can. As the gas is released from inlet pipe distal end 162 into the liquid medium 110, bubbles 180 are formed and released to the top portion of can 120. Outlet pipe 154 collects the gas from the top portion of the can and directs the gas back towards compressor 152.

Also during operation, HV power source 170 is activated to generate HV, which is supplied to electrode 172 via electric cord 174. The HV at the pointed tip 176 applies a plasma-generating EM field between the pointed tip and the surface of the liquid medium at the inlet pipe distal end 162. Streaming of air through the liquid via bubbles allows for increased rate of activation of the liquid, due to increased surface area of the liquid that is exposed to plasma. Also, due to the constant streaming of air and formation of bubbles, the liquid is agitated and different portions of the liquid are exposed to the plasma over time. Hence the risk of heating the liquid medium to an undesired level may be prevented.

In some embodiments, it may be advantageous to actively remove heat from the can by cooling, during plasma activation. In some such embodiments, the circulating system 150 may comprise a cooling module 180 for cooling the gas circulated by the circulating system. Such a cooling module may include a radiator and/or a heat exchanger, and/or a compressor (all not shown in the figure). In some embodiments, cooling the circulating gas may be carried out directly, rather than by heat exchange with a coolant fluid such as using a radiator—by compressing the circulating gas and releasing the compressed gas via an aperture.

In some embodiments, the circulating system 150 is not a closed system. In an open circulating system (not shown here), an outlet port of can 120 may release to the ambient excited gas, after the gas has gone through the liquid medium in can 120. Furthermore, in an open circulating system, compressor 152 may collect air from the ambient and not from an outlet pipe fluidly associated with the can.

In such embodiments that employ an open circulating system, cooling the circulating gas may be carried out directly by compressing the gas through a vortex tube. It is noted that during operation, a vortex tube releases—simultaneously with an output cold stream—an output hot stream, which may preferably be released to the ambient. Thus, the use of a vortex tube for cooling is made simpler in an open circulating system.

In some embodiments described herein, where gaseous residuals of plasma excitation are released to the ambient, an active filter may be employed to capture hazardous species and prevent the release of such species to the atmosphere. The active filter may be positioned up-stream from the point of release of gas to the ambient—e.g., downstream from the hot exhaust of a vortex tube—so that all gas passes through the active filter prior to being released to the atmosphere.

In some embodiments, prevention of gelation due to heating during plasma activation may be carried out by cooling the liquid medium. However, this approach may, in some circumstances, be inferior to cooling the circulating gas. Cooling the circulating gas removes heat from the source, namely from the region where plasma is generated, and therefore it may be, in some circumstances, more efficient.

FIG. 1E schematically depicts an embodiment of an inlet tube 190 and a hollow needle 192 employed as an electrode and configured to electrically associate with a HV power source (e.g., power source 170 of FIG. 1D). Hollow needle may be embodied, for example, by a syringe needle. Hollow needle 192 is sealed inside inlet tube 190 by a needle seal 194. When air is compressed into inlet tube 190 as is explained above, the compressed air is forced into hollow needle 192 to be ejected from a needle distal end 196. Thus, in the embodiments of FIG. 1D and FIG. 1E the plasma-generating EM field is applied directly to the stream of gas flowing onto the liquid medium.

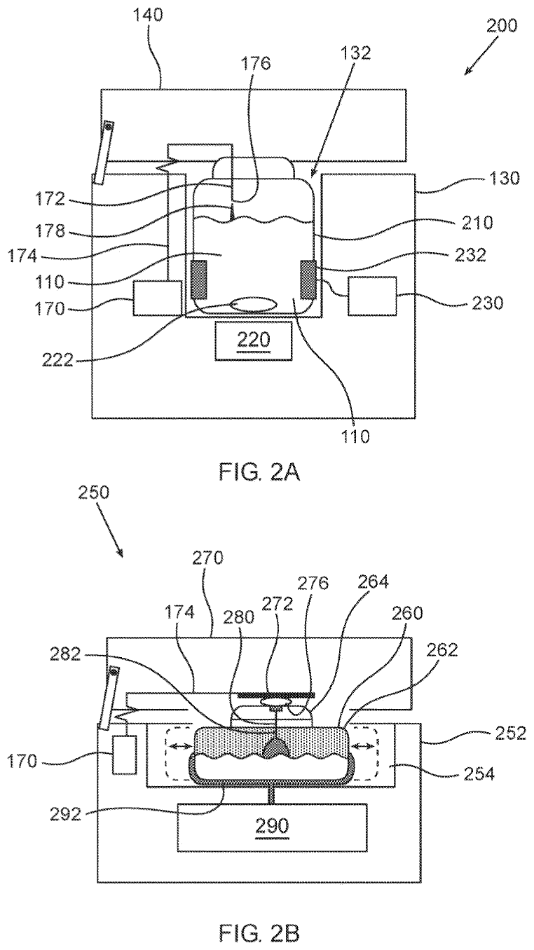

FIG. 2A depicts another embodiment of a device 200 for plasma activating liquid medium 110 in a can 210. Device 200 comprises housing 130 with slot 132 and door 140 configured to close onto the slot. Device 200 further comprises HV power source 170 electrically associated with electrode 172 via electric cord 174. In some embodiments, device 200 may be devoid of a gas circulating system. Device 200 is different from device 100 in that device 200 employs stirring for mixing the liquid composition during plasma activation, thereby preventing heating and increase of temperature in one region of the liquid composition, thus preventing gelation of the liquid composition during plasma activation. Stirring also promotes homogenous activation across the liquid. Device 200 thus comprises a magnetic stirrer 220 configured to generate a revolving magnetic field inside slot 132. Can 210 comprises a magnetic slab 222 adapted to react to a magnetic field. Magnetic slab 222 may be made, for example, of a ferromagnetic material such as iron, of a permanent magnet, and may be coated with an inert material such as plastic to prevent oxidation or other chemical interaction with the liquid composition.

For use, a cover of can 210, used to cover the can during storing and transportation, may be removed by the user, the can may be inserted into slot 132 and door 140 may be closed. Closing the door introduces electrode 172 into can 210 wherein pointed tip 176 is advantageously above the liquid surface. When HV power source 170 is activated, a plasma generating EM field is applied between the pointed tip 176 and the liquid surface, thereby generating plasma in the region therebetween. During plasma activation, magnetic stirrer 220 may also be activated, thereby effecting revolving of magnetic slab 222 and stirring of the liquid medium 110.

It is noted that—in some embodiments—the maximum power which may be employed through a single electrode may be limited by a local temperature rise in the vicinity of the electrode. An attempt to increase the power beyond such a limit may cause local temperature rise of the liquid medium, and cause, for example, gelation in a liquid composition capable of undergoing a liquid-gel phase transition, which may be undesired. Thus, in some embodiments, a device for plasma activating a liquid medium may comprise two electrodes instead of one. Such a device with two electrodes may have an advantage of allowing employing higher power for plasma activation, thus reaching a given level of activation, or a given concentration of RONS, in a shorter time compared to a device with a single electrode.

For example, a device such as device 200 may comprise two electrodes such as electrode 172, mutatis mutandis. In some such embodiments, the device may comprise two HV power sources, so that each electrode may be electrically associated with a different HV power source. Consequently, each of the two electrodes may generate a plasma activating EM field independently of the other electrode. In some embodiments two electrodes may be electrically associated with a single HV power source. In some such embodiments the two electrodes may be supplied with HV simultaneously, whereas in some embodiments a HV distributor may distributed the HV to the electrodes sequentially.

Advantageously, the electrodes may be arranged so that the distance between each electrode's distal end (e.g., pointed tip 176 in FIG. 2A) to the liquid surface is shorter than the distance between the electrodes and shorter than the distance between each electrode and the wall of can.

In some embodiments, device 200 further comprises a chiller 230 configured to remove heat from can 210 and the liquid medium therein. Chiller 230 may be thermally associated with can 210 via heat exchangers 232. In some embodiments, chiller 230 may be activated during plasma activation so as to prevent or decrease temperature rise in the liquid composition and prevent gelation. In some embodiments vapor-compression cooling may be employed. An advantage of conventional vapor-compression cooling in a closed cycle comprising a compressor and an evaporator, is relatively high efficiency; possible disadvantages are large dimensions and relatively long time to reach a target temperature. In some embodiments thermoelectric coolers (TEC) may be used. Thermoelectric coolers, using, for example, Peltier cooling, may have an advantage of relatively small dimensions, lack of moving parts and simple adaptation to a desired shape of the object to be cooled (e.g., can 210). In some embodiments a passive chiller may be used as is detailed further below.

It is note that a chiller such as chiller 230 and/or a stirrer such as stirrer 220 (and a corresponding magnetic slab 222) may be used, mutatis mutandis, with any of the embodiments described herein, e.g., FIGS. 1A-1D, FIG. 2B etc.

FIG. 2B schematically depicts yet another embodiment of a device 250 for plasma activating liquid medium 110 in a can 260. Device 250 employs vigorous shaking of can 260 during plasma activation, for agitating and mixing the liquid medium, during plasma activation. In some embodiments such vigorous shaking may generate spray 262 inside the can. In some embodiments the liquid medium may transform, partly or in whole, to foam.

Device 250 comprises a housing 252 with a slot 254 and a door 270 configured to close onto the slot. Device 250 further comprises HV power source 170 electrically associated with a sliding pad 272 via electric cord 174. Can 260 comprises a removable can cover 264, comprising a sliding contact 276 on the outside surface of the can cover. Sliding contact 276 is electrically associated with an electrode 280 attached to the inside surface of the can cover and having a pointed tip 282. In some embodiments, the sliding contact 276 is in electrical contact with electrode 280 via a feedthrough (not shown here) in can cover 264. In some embodiments, can cover 264 is made of a dielectric material. In some embodiments, can cover 264 is metallic and electrode 280 is electrically isolated from the can cover. In some embodiments, can cover 264 is metallic and is electrically connected to the electrode.

Device 250 further comprises a vibration generator 290 mechanically associated with a can holder 292. For use, can 260 may be positioned in slot 254 and attached to can holder 292. When door 270 is closed, sliding contact 276 contacts sliding pad 272, thereby electrically associating electrode 280 with HV power source 170. During plasma activation, vibration generator 290 is also activated for strongly vibrating or vigorously shaking can 260. Activation of power source 170 generates plasma over a relatively large region around pointed tip 282, presumably because of generation of a multitude of discharging trajectories between the electrode and the liquid composition, due to formation of spray 262 in the space around the pointed tip. The interface of plasma with the multitude of droplets in the spray increases considerably the surface area of liquid composition in contact with gaseous plasma, and expedites activation of the liquid. The mixing of the liquid due to the shaking of can 260 further prevents heating and increase of temperature in one region of the liquid composition, thereby preventing gelation, if the liquid medium is the liquid composition of the invention, during plasma activation. After plasma activation, the user may remove the can from the can holder 292, remove the can cover 264, and use the liquid medium as desired.

In some embodiments, additionally or alternatively, vibration generator 290 may apply rotational displacements to can 260. In some such embodiments, such rotational displacements may be back and forth. In some such embodiments, can 260 may comprise fins or blades (not shown here) to assist in mixing the liquid medium during such rotational displacements.

In some embodiments, device 250 may further comprise a cooler or a chiller (not shown here) for cooling the liquid composition during plasma activation. In some embodiments, such cooler may be thermally associated with can 264 via the can holder 292 or via a dedicated heat exchanger in thermal contact with the can.

### Example 4

### a Transportable Sealed can Containing Liquid Composition

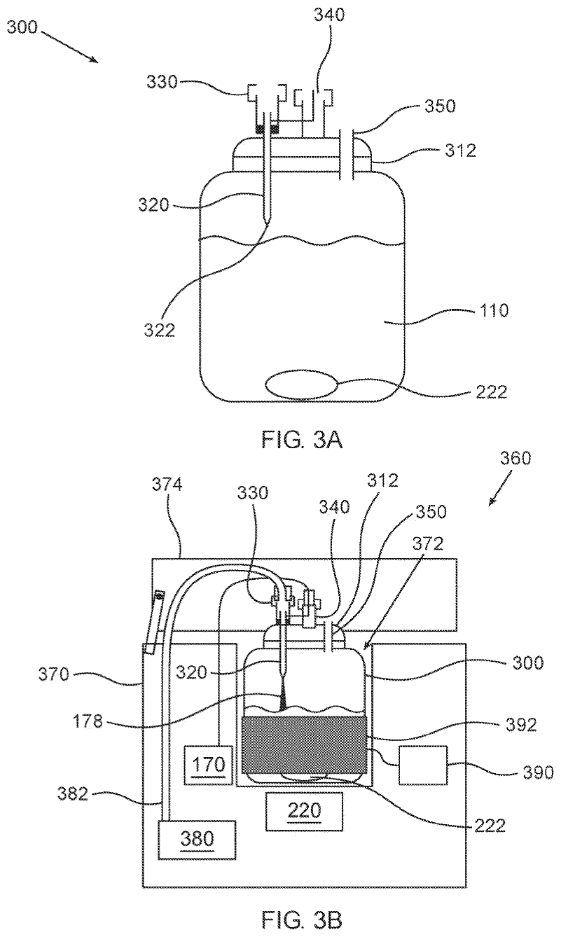

FIG. 3A illustrates schematically an embodiment of a sealed can 300 comprising liquid medium 110 according to the teachings herein. Sealed can 300 is configured for storing and/or transport, and for use in a device for plasma activating the liquid medium according to the teachings herein. In some embodiments sealed can is substantially made of a dielectric material such as glass or plastic. In some embodiments sealed can 300 is made from metal. In some such embodiments, the can walls may be electrically associated with ground potential during plasma activation.

Sealed can 300 is configured to be hermetically sealed during storing or transport so that liquid medium 110 may not escape the sealed can, and also may not be contaminated by contaminants entering the sealed can from the outside. In some embodiments sealed can 300 may comprise a cover 312 for sealing the can's opening. In some embodiments cover 312 may be untighten by a user so as to fluidly connect the inside of sealed can 300 with the ambient. In some embodiments cover 312 may be removed from the can's opening altogether.

Sealed can 300 comprises a hollow needle 320, e.g., a syringe needle. Hollow needle 320 is electrically conducting being typically made of metal and having, in some embodiments, a pointed distal end 322. Sealed can 300 further comprises a gas port 330 in flow communication with the lumen of the hollow needle. Thus, gas port 330 enables flowing gas into the can through hollow needle 320. Gas port 330 is sealable thus being configured to be sealed when the sealed can is in storage or during transport, and may be opened for gas flow by a user, e.g., during plasma activation as described below.

Sealed can 300 further comprises a HV connector 340 electrically associated with hollow needle 320. HV connector 340 is configured to electrically associate high voltage to the hollow needle. Upon receiving HV, e.g., form a HV power source, the hollow needle may apply a plasma-generating EM field inside the sealed can, preferably between the distal end 322 and the surface of the liquid medium 110. In some embodiments plasma is generated by the hollow needle in the arcing mode.

In some embodiments a gas port and a HV connector may be embodied in a single connector.

In some embodiments sealed can 300 comprises an exhaust port 350 configured to enable flowing gas from the sealed can to the ambient. Exhaust port 350 may be sealed during storing and/or transport of sealed can 300, and may be opened for gas flow by a user, e.g., during plasma activation as described below. In some embodiments exhaust port 350 may be sealed by a foil which may be removed or torn to open the port. In some embodiments exhaust port 350 may be configured to be fluidly associated to a gas flow channel (not shown here), e.g., a tube, to direct the outflowing gas to further processing, e.g., for filtering prior to releasing the gas to the ambient, or for re-circulating, as described in FIG. 1D above. In some embodiments the exhaust port 350 may be configured to release the outflowing gas to the ambient without further processing. In some embodiments outflowing gas may be released to the ambient via an opening of the sealed can, e.g., by way of untightening the cover 312.

In some embodiments sealed can 300 comprises a magnetic slab 222, allowing stirring the liquid composition by a magnetic stirrer, as described above in FIG. 2A. Additionally or alternatively, the sealed can may comprise fins or blades (not shown here) to assist in mixing the liquid medium during shaking or rotating the can, as described above in FIG. 2B.

Sealed can 300 may be used in conjunction with a device 360 for plasma activating a liquid medium, as depicted in FIG. 3B. Device 360 may comprise a housing 370 and a slot 372 in the housing, configured to accept the sealed can as described above for, e.g., FIG. 2A. A door 374 may be employed to lock sealed can 300 in the slot.

The sealed can may be manufactured—preferably under sterile conditions—and properly stored. When required, e.g., by a clinic, the sealed can may be transported to the clinic. In the clinic the sealed can may be placed in slot 372 of the device for plasma activation. Gas port 330 may be connected to a gas flow source, e.g., a compressor 380, via an inlet channel 382. HV connector 340 may be electrically connected to HV power source 170. A gas outflow from the can may be enabled, e.g., by opening exhaust port 350, or, for example, by untightening cover 312 of the sealed can. In some embodiments exhaust port 350 may be fluidly connected to an outlet channel of the device, that may include a filter (both are not shown here) for absorbing or removing hazardous residuals of the plasma before releasing the gas to the ambient.

Plasma activation of the liquid composition may then be effected by supplying HV to the hollow needle 320, preferably while flowing gas through the hollow needle into the sealed can, and while agitating the liquid composition, e.g., stirring the liquid using the magnetic slab 222 and magnetic stirrer 220 of the device, or using any other method. An active chiller 390 may be employed to maintain the liquid composition at a sufficiently low temperature, using heat exchangers 392. According to some embodiments a passive chiller may be employed as described herein further below.

After plasma activation is complete, the sealed can may optionally be removed from slot 372. The sealed can may be opened, e.g., by removing cover 312 from the can's opening, thus exposing the activated liquid medium therein. The activated liquid medium may then be taken for application, e.g., by drawing the liquid into a syringe and then applying the liquid to a region in need of the body.

### Example 5

### Using a Spray Nozzle

According to an aspect of the invention a spray nozzle may be used to apply activated liquid composition of the invention onto a body part. In some embodiments the liquid composition may be poured into a spray bottle and the spray bottle may then be used for applying the material. In some embodiments a spray nozzle head (e.g., such as that of a spray bottle) may be assembled onto the can that was used for plasma activating the liquid. In some embodiments the spray nozzle head may comprises a positive displacement pump—possibly, but not necessarily manual—for dispensing the material through the nozzle thus generating spray. For example, sealed can 300 may be used to plasma activating the liquid composition therein, as described above, following which cover 312 may be removed and a spray nozzle head may be attached to the can, e.g., screwed onto the can, to be used for applying the liquid composition onto the body part.

### Example 6

### Foaming the Liquid Composition

In some embodiments, foaming the liquid composition of the invention may transform the liquid into a relatively stiff foam. “Relatively stiff” here means foam which is at least as viscous as the gel according to the teachings herein.

In some embodiments, such foaming of the liquid may require intense agitation of the liquid composition—e.g., fast stirring—or a combination of intense agitation and strong air flow onto the liquid composition (for example, from the hollow needle). The terms “intense agitation” and “strong air flow” herein are used in a relative sense, meaning that agitation or flow intensity must be above respective thresholds to generate foam. Thus, there is provided according to an aspect of the invention, a plasma-activated foam which may be generated by plasma-activating the liquid composition while intensively agitating the liquid and possibly while flowing onto the liquid a strong gas flow.

It has been found by the inventors that an exemplary liquid composition of the invention made by mixing 20.00% (wt.) Poloxamer 407 with 5.00% Poloxamer 188 and 75% water in a total amount of 10 milliliter, transformed into foam following mixing, using a magnetic stirrer, at a rate of above 1000 RPM for 3 minutes while being maintained at a temperature lower than 5 degC. The foam was stable for about 10 minutes at temperature below 5 degC., after which it turned back to a liquid form.

It is noted that a plasma-activated foam may be highly useful because the liquid-foam transformation hardens the material. In other words, the generated foam may be viscous enough to allow its easy application to a body part—e.g., a portion of the skin—and allow the material's stability over the location of application, without risk of running or flowing of the foam. In some embodiments such hardening of the material due to foaming is possible regardless of a capacity of the material to undergo a liquid-gel phase transformation. In such embodiments the liquid composition used for plasma activation should not necessarily be able to undergo a liquid-gel phase transition, and hence the liquid composition may be simpler to manufacture and/or cheaper (compared to a liquid composition which is capable of undergoing such a phase transition). However, it should be understood that a liquid composition according to the teachings herein which does undergo a liquid-gel phase transition may be advantageous even when used as a foam, due to its ensured stability over long periods of time, e.g., several days.

### Example 7

### Passive Chiller

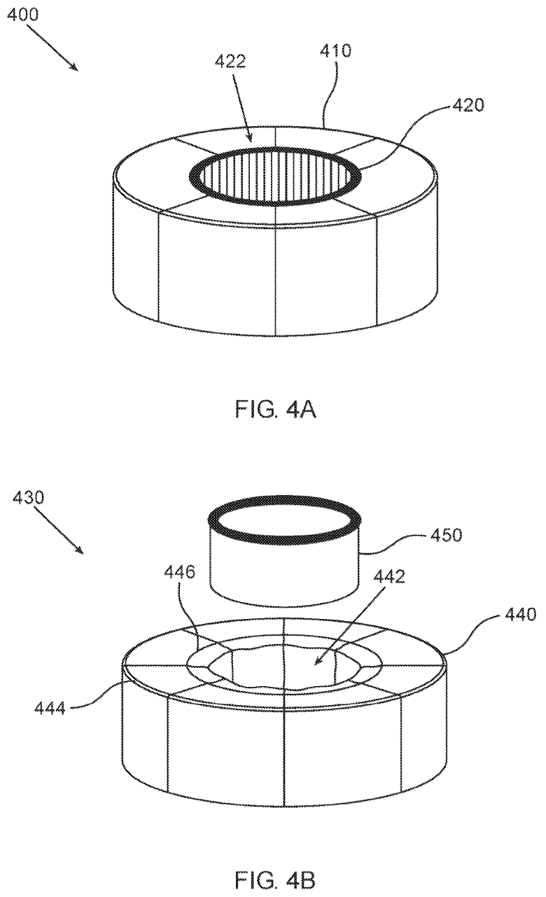

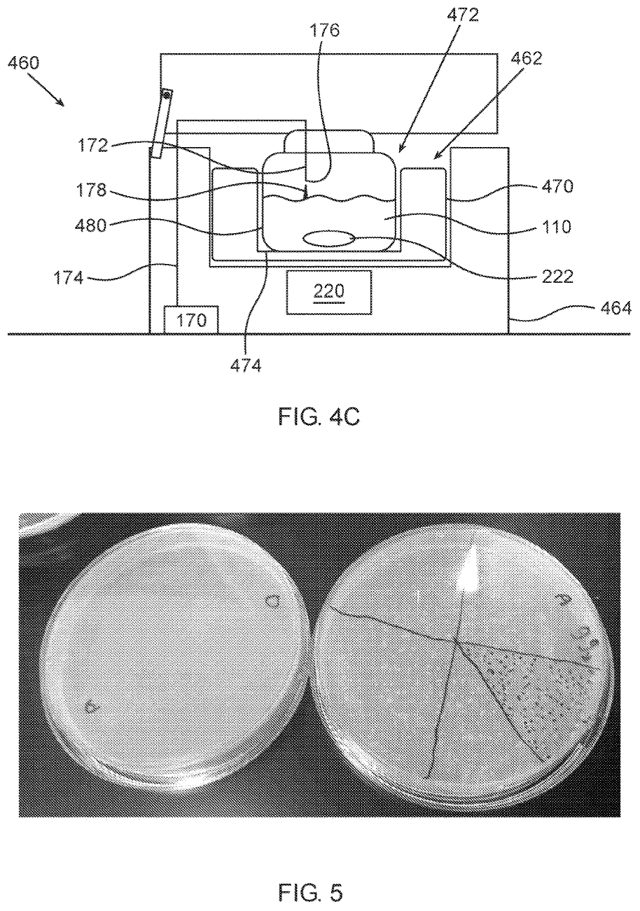

FIGS. 4A and 4B illustrate schematically embodiments of passive chillers that may be used, according to aspects of the invention, with the devices for plasma activating a liquid medium as described above.

FIG. 4A illustrates an embodiment of a passive chiller 400 comprising an ice-pack 410 in a form of a torus, surrounding a can 420. In some embodiments can 420 may be open on top, as depicted; in some embodiments can 420 may be closable by a cover (not shown here). In some embodiments can 420 is detachable from ice-pack 410. In such embodiments the ice-pack has a chiller slot 422 dimensioned and configured to house the can when the can is inserted into the slot. Advantageously, chiller slot 422 is dimensioned and configured to establish good thermal contact of the ice-pack with the can. In some embodiments a flexible metallic member (not shown here) inside slot 422 adapts to the external shape of can 420 when can 420 is inserted into the slot, so as to establish good thermal conductance between the ice pack and the can. In some embodiments the ice-pack and the can are attached together, the walls of the can functioning also as walls of the ice-pack.

Embodiments of ice pack 410 may have a great variety of shapes and forms, as can be realized by a person skilled in the art. The external shape thereof may be round as depicted in FIG. 4A or rectangular or any other shape as the case may be. In some embodiments ice-pack 410 may also extend in a region underneath can 420, so as to extract heat from can 420 not only through the side walls of the can but also from the can's bottom wall (the can's floor).

Ice pack 410 contains a coolant material having a melting temperature of around zero degrees Celcius, e.g., between about −10 degC. and about +5 degC. Advantageously, but not necessarily, the material may have a relatively high latent heat of fusion (referred to herein as ‘latent heat’ for short), e.g., above 200 J/gr or above 250 J/gr or even above 300 J/gr. For example, ice pack 410 may be filled with water, having a freezing temperature of about 0 degC. and latent heat of about 333 J/gr. Additionally or alternatively, ice-pack 410 may be filled with a gel material such that may be often found in commercial ice-packs for home use, such gels being obtained, for example, by adding to water additives such as hydroxyethyl cellulose, sodium polyacrylate or vinyl-coated silica gel.

Ice pack 410 may be made of a rigid or semi-rigid material. Optionally, ice pack 410 may be made substantially of plastic, like a commercial ice-pack for home use. Can 420 may be made of a stiff dielectric material or of an electrically conducting material such as metal.

FIG. 4B is an exploded view of an embodiment of a passive chiller 430. Chiller 430 comprises ice-pack 440 encompassing a chiller slot 442, and a detachable can 450 dimensioned and configured to be inserted into chiller slot 442. Ice-pack 440 is different from ice pack 410 of chiller 400 in having an external chamber 444 and an internal chamber 446—encompassed by the external chamber—for two different materials, respectively. Advantageously, external chamber 444 stores a coolant material having a freezing temperature around zero degrees c. and preferably a high latent heat, as described above for ice-pack 410. Internal chamber 446 stores a material that remains in a liquid phase at temperatures lower than the freezing temperature of the coolant in external chamber 444. In some embodiments the material in internal chamber 446 may remain liquid at temperatures lower than −10 degC., even more preferably at temperatures lower than −24 degC. Advantageously, the liquid in internal chamber 446 has a relatively high thermal conductance. For example, the liquid is chamber 444 may be distilled water with antifreeze additive such as ethylene glycol or propylene glycol or the like.

Internal chamber 446 may advantageously be made from a soft or flexible material. Thus, when can 450 is inserted into slot 442, the liquid content of the internal chamber, together with softness of its walls, ensure adjustment of the slot to the external surface of the can, thus ensuring good thermal contact between the internal and the can.

FIG. 4C illustrates a device 460 for plasma activating a liquid medium which incorporates a passive chiller 470 as described herein, positioned in a slot 462 of a housing 464 the device. A detachable can 480 is shown in a chiller slot 472 being in thermal contact with a cold portion 474, e.g., the ice pack, of the passive chiller. Device 460 further comprises electrode 172 electrically associated with a HV power source 170, and configured to apply a plasma generating EM field between the electrode and the surface of the liquid medium 110. The device further comprises a magnetic stirrer 220 as described above.

Prior to operation of the device, the ice pack of the passive chiller may advantageously be cooled down to below freezing temperature of the coolant. When the coolant is frozen, the ice pack may be placed in the slot 462 and the can 480 may be positioned in the chiller slot 472.

According to an example, 50 cc of liquid composition at an initial temperature of about 0 degC. are plasma-treated in the device 460. The power flowing into the liquid composition through the plasma-generating electric field, through stirring and by way of heat diffusion from the ambient totals about 100 W. An ice pack containing about 300 cc of coolant having latent heat of about 330 J/gr, may absorb from the liquid composition the associated heat, before melting fully, for a duration of more than 15 minutes. Heat transfer from the liquid composition to the coolant occurs across the interfacing surface, namely the can walls and possibly the walls of the ice-pack. A total of interfacing surface of about 150 cm\hat{ }2 and total heat conductance of about 50 W/degC*m, enables a temperature gradient of less than 5 degC across the interfacing surface.

After the plasma treatment is complete, the activated liquid medium may be taken for use, whereas the ice pack may be taken to the refrigerator, to freeze the coolant.

### Example 8

### Treating Skin Disorders

PAM prepared by any of the methods described above may be used to treat skin disorders other than otitis externa in pets and other domestic animals. Such skin disorders may be caused by yeast or fungi such as Malassezia (causing Malassezia dermatitis) and Ringworm or by bacteria (e.g., Staphylococcus intermedius pyoderma, or Alabama rot caused by E. coli toxins). Skin disorders may also be caused by parasites, such as Sarcoptic mange (scabies), mange caused by Demodex mites (Demodicosis) and Cheyletiella. Viral skin disorders, such as warts, may also be treated according to the teachings herein. According to some embodiments, PAM of the invention may be used to treat skin disorders attributed to autoimmune system dysfunction combined with external or environmental triggers, for example dermatitis, e.g., atopic dermatitis. According to some embodiments, PAM of the invention may be used to treat skin disorders in humans.

To employ PAM on a bare skin according to the teachings herein, a liquid composition with similar viscosity or higher viscosity (compared to a liquid composition prepared for treating ear infections as described above) may be prepared and activated by plasma. The liquid composition may then be applied directly onto the skin or used to soak a gauze or a bandage which are applied to the skin. In some embodiments the liquid composition may be applied to the skin by spraying as described above. In some embodiments the liquid composition may be foamed and then applied to the skin. The foam's stiffness may assist in such cases to ensure stability of the activated composition in site on the skin, until the material transforms to gel.

In some embodiments, the liquid composition may gel as described above, in response to attaining an elevated temperature, e.g., body temperature. In some embodiments, gelation may be achieved by mixing the activated liquid composition with a cross-linking agent. Additionally or alternatively, cross-linking may be achieved by exposing the PAM to visible or UV light (see for example Lim K. S. et al, “Visible Light Cross-Linking of Gelatin Hydrogels Offers an Enhanced Cell Microenvironment with Improved Light Penetration Depth” Macromolecular Bioscience, vol. 19, no. 6, 1900098, htps:doi.org/10.1002/mabi.201900098).

### Example 9

In Vitro Reduction of Bacterial Load Taken from an Infected Ear

A composition as outlined in Table 1 was activated by plasma using the stirrer mode (Example 2, FIG. 2A). The plasma-activated composition was applied to secretions derived from an infected ear of a dog that were incubated on an agar plate. A reduction of up to 3.5 logs in bacterial load was observed when using an activated gel (FIG. 5, left plate) as compared to the control (FIG. 5, right plate).

### Example 10

### Pam in Treatment of Periodontal Infection

Periodontal disease is a common chronic inflammatory condition all over the world. Often, poor or ineffective oral hygiene cause gingivitis, namely inflammation, expressed in redness and swelling of the gingiva. In some cases, gingivitis may progress to periodontitis—a bacterial infection which stimulates a host response resulting in the loss of the supporting structures (soft and hard tissues) of the tooth. With the destruction of the gingival fibers, the gum tissues separate from the tooth resulting in a periodontal pocket. Periodontitis is one of the prevailing reasons for adult tooth loss if left untreated. It is a disease which is difficult to treat, requiring professional sub-gingival mechanical cleaning termed scaling and root planning (SRP). If non-surgical treatment is not successful, surgical therapy may be necessary. SRP is often accompanied by local antimicrobial treatment at the infected site or by systemic antibiotics. An example of a local antimicrobial treatment not based on antibiotics is provided by a slow-release chlorhexidine chip which is positioned by the treating dentist in the periodontal pocket (see for example https://www.rxlist.coim/periochip-drug.htm#description and https://www.periochip.com). The chip releases chlorhexidine in a biphasic manner, releasing approximately 40% of the chlorhexidine within the first 24 hours and then releasing the remaining chlorhexidine approximately linearly for 7-10 days.

Sub gingival destructive inflammation may sometime evolve in soft tissues surrounding dental implants (peri-implantitis). As a result, alveolar bone (hard tissue), which surrounds the implant for the purposes of retention, may be lost over time. Peri-implantitis and Periodontitis may thus be treated according to the teachings herein which, for the sake of simplicity, refer to periodontal pockets.

According to some embodiments of the current invention, a plasma-activated medium (PAM) may be applied into a periodontal pocket of a subject in need thereof. When in the periodontal pocket, the PAM gradually releases RONS, promoting disinfection of the pocket's surroundings. The PAM is biodegradable and following a pre-determined period, typically of a few days, the PAM decomposes and its remains are naturally washed away from the pocket.

In some embodiments, the PAM may be a composition comprising a synthetic biocompatible polymer capable of in-situ gelation. A preferred stimulus of gelation may be an elevation of temperature from typical room temperature (e.g., about 26° C.) to typical body temperature (e.g., about 37° C.). The liquid composition may be activated by plasma as described above in EXAMPLE 2. The resulting PAM may then be applied to the periodontal pocket, where the composition transforms into gel and stabilizes in situ. According to some embodiments, the liquid composition substantially fills the periodontal pocket, thereby effectively preventing food debris and other potential contaminants from entering the pocket.

Due to the wet surroundings of the periodontal pocket, appropriate measures should be taken to prevent the resulting gel from dissolving. In some embodiments, a higher concentration of poloxamer (compared to concentrations required for gelation in dry surroundings) may suffice for providing a suitably stable gel.

In some embodiments, a further hardening mechanism may employ optical cross-linking. Optical cross-linking may employ UV light or visible light. In such embodiments, the liquid composition may be injected to the periodontal pocket and then be cross-linked by exposing to light. Light may be applied via an optical fiber inserted gently into the periodontal pocket and scanning the top surface of the filling. In some embodiments, the liquid composition may transform to gel having a first level of hardness or viscosity, in response to an elevated temperature as is explained above, and the gel may then be further hardened or solidified to a higher level of viscosity, by light-induced cross-linking.

In some embodiments, a liquid composition may be plasma activated as described above and then may be solidified outside the periodontal pocket, in a mold. The resulting molding, possibly in a form of a thin slab, may be inserted into the periodontal pocket and fixed there inside. Solidifying the PAM outside the periodontal pocket has the advantage of employing solidifying and hardening procedures that may enable reaching a desired level of hardness but may not be employed when the PAM in inside the patient's mouth, such as chemical crosslinking. However, it may have a disadvantage of yielding PAM in predetermined shapes and sizes only, namely such that cannot fill the periodontal pocket completely.

### Example 11

### Treatment of Pre-Malignancies and Malignancies

Pancreatic cancer is the third-most-common cause of death from cancer in the U.S. Because the pancreas is located deep in the body, behind the stomach, it is not easily accessible to palpation and a tumor might often be detected at a relatively late stage, when the tumor growth has affected the normal functionality of the pancreas itself or other near-by organs. Even when a tumor is detected at a relatively early stage, the one-year and five-years survival rates are relatively low, because surgery is currently the only medical procedure that may lead to cure, and because, due to the pancreas' location and other factors, surgery is complex and not always possible.

A fraction of pancreatic cancers evolves from cysts, although most pancreatic cysts will not become cancerous. This state of affairs generates a dilemma as to how to treat a pancreatic cyst once it is detected. Generally, the sequence of operations includes defining the type of cyst to determine whether it might be cancerous or not, and obtaining a biopsy in suspicious cases. If the biopsy indicates a non-cancerous or precancerous cyst, the advocated strategy is often watchful waiting, in an attempt to avoid a complex surgery to remove a cyst, that, more often than not, will not become cancerous within a short while even if left untreated. It is evident, however, that such a watchful waiting period is necessarily causing both a burden and a risk.

There is thus provided, according to an aspect of the invention, a method of treating a pancreatic cyst by PAM, according to the teachings herein. The method is aimed at reducing or eliminating inflammation at and around the cyst, thereby eliminating the cyst itself. Additionally or alternatively, the method is aimed at selectively reducing the number of precancerous cells or even eliminating precancerous cells in the cyst. The method comprises delivering a plasma-activated composition, according to the teachings herein, to the cyst (or lesion or tumor). The plasma-activated composition is in a liquid form during delivery, and it gels following deployment in the patient's body.

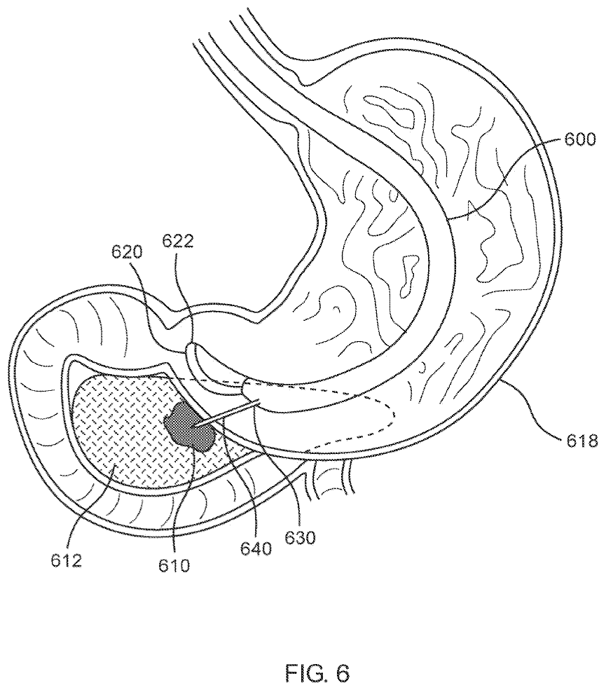

FIG. 6 schematically depicts an embodiment of an endoscope 600 employed to deliver a plasma-activated liquid composition into a cyst 610 in a pancreas 612 of the patient. The endoscope is advanced through the patient's mouth and esophagus into the patient's stomach 618 (depicted here in cross-section view). The endoscope may conveniently comprise an ultrasound transceiver 620 at a distal end 622 thereof, the ultrasound transceiver being functionally associated with an ultrasound device comprising a screen positioned outside the patient's body and configured to provide to a medical practitioner an ultrasound image of the surroundings of the transceiver 620. The endoscope further comprises a work channel configured as a lumen passing through the entire length of the endoscope. The work channel enables a practitioner to advance a working tool through the work channel and through an exit opening 630 near the distal end 622, and to maneuver the working tool using a control unit outside the patient's body, as is well known in the art.

To deliver the plasma-activated composition to the cyst 610, the practitioner may employ the ultrasound transceiver 620 to search and detect the cyst 610 and to position the distal end 622 in an appropriate location relative to the cyst to perform the injection. The practitioner may then advance an injecting needle 640 through the work channel and the exit opening 630 of the work channel. The injecting needle is in flow communication with an injecting syringe located outside the patient's body, via an elongated tube (both are not shown here) that passes through the work channel. Prior to operation, a liquid composition may be plasma activated as described for example in EXAMPLEs 2-7 above and the injection syringe may be filed with the plasma-activated composition in the liquid form. By maneuvering the injection needle, the practitioner may penetrate the pancreatic cyst through the stomach wall. Further, by using the injection syringe, the practitioner may inject the plasma activated composition through the tube and the injection needle into the cyst.

According to some embodiments, the work channel may be constantly washed with cold saline during the procedure, to prevent the liquid composition in the tube inside the work channel to gel prematurely. The saline may be cooled down to about 5° C. and may be flown through the work channel to be expelled through the exit opening 630 into the stomach.

According to some embodiments, the cyst may be drained, e.g., through the injection needle 640, prior to injecting the composition into the cyst.

### Example 12

### Treatment of Post-Malignancies—Tumor Excision in the Bladder

Following tumor excision in the bladder or colon, a plasma-activated composition according to certain embodiments of the present invention is applied to the excision site in order to prevent tumor recurrence.

Transurethral resection f bladder tumor (TURBT) is the gold standard for surgical treatment of bladder cancer. Unfortunately, the post-surgery recurrence rate of bladder cancer is high. Current standard of care to further reduce bladder cancer recurrence is instillation of intravesical chemotherapy (ICT). An alternative that has been suggested is irrigation of the bladder with saline or with water (See A. Mahran et al., “Bladder irrigation after transurethral resection of superficial bladder cancer: a systematic review of the literature” Can. J. Urol. 25(6), Dec. 2018 P. 9579).

According to an aspect of the invention, plasma-activated composition according to the teachings herein may be used for post-surgery treatment, to decrease or eliminate the likelihood of cancer recurrence. According to an embodiment. an aqueous solution, e.g., saline, is plasma-activated by exposing the solution to gaseous plasma. According to some embodiments, the aqueous solution is exposed to plasma generated in air at room pressure. According to some embodiments, the gaseous plasma is generated in a dielectric barrier discharge (DBD) mode by applying an electromagnetic field between an anode located above the surface of the solution and a cathode located beneath the solution or electrically coupled with the solution. According to some embodiments, the anode is coated with an electric insulation layer to provide the DBD mode of generating plasma. In some embodiments, a line of sight between the anode and the surface of the solution is interrupted by a dielectric layer for the same.

According to some embodiments, the aqueous solution is plasma activated by a plasma jet. To this end, a plasma generator may be positioned above the surface of the solution and a stream of gas may be steered through the plasma generation region of the plasma generator, to be excited by plasma. The excited stream of gas is then focused to form a jet directed towards the surface of the solution. According to some embodiments, the stream of gas includes an inert gas such as helium or argon or a gaseous mixture comprising the same.

According to some embodiments, the solution may be stirred during exposure to the gaseous plasma to expedite the dissolving of plasma excitation products in the solution.

After the aqueous solution is plasma activated, e.g., as described above, the plasma activated solution may be used to irrigate the bladder. In some embodiments, bladder irrigation is performed as is known in the art, using a catheter passing through the urethra. According to some embodiments, the plasma activated solution is maintained in the bladder for a period between 1 minute and 2 hours, including each value within the specified range.

### Example 13

### Treatment of Post-Malignancies—Polyp Excision in the Colon

Rectal villous adenoma is a type of colon polyp (out of five types that are usually considered) that makes about 15% of polyps detected in colon cancer screening tests, and carries a high risk of turning cancerous. About 40% of cases of complete excision procedures where the samples were discovered in pathology to be cancerous, experience recurrence (see for example Sungeyun David Cho, “Treatment Strategies and Outcomes for Rectal Villous Adenoma From a Single-Center Experience”, Arch Surg., 143(9), 2008, P. 866).

According to an aspect of the invention, plasma-activated composition according to the teachings herein may be used for post-surgery treatment, to decrease or eliminate the likelihood of cancer recurrence. A liquid composition, capable of gelling at or near body temperature, is plasma-activated, e.g., as described in EXAMPLE 2 above. The method of application of the composition to the desired location is similar to that described above vis a vis FIG. 6. An injector fluidly associated with an elongated tube is filled with the composition. A colonoscope may be steered through the colon until the colonoscope's distal end is proximal the surgery region and the elongated tube is impelled along a work channel of a colonoscope. Then the composition is injected via the tube onto the region of surgery where it gels on the walls of the colon. A stent may be positioned in the place where the composition is applied, to protect the gel on the colon walls from being washed way.

There is thus provided according to an aspect of the invention a system (capsule 10 in activation unit 60, FIG. 1B; devices 100, 200, 250, 360, 460 in FIGS. 1D, 2A, 2B, 3B and 4C respectively) for providing plasma activated medium (PAM) for medical use. The system comprises a vessel (10, 120, 210, 260, 300, 480) configured to contain a liquid medium (12, 110). The system further comprises an electrode (38 in FIG. 1B, 172 in FIGS. 1D, 2A and 4C, 192 in FIG. 1E, 280 in FIG. 2B, and 320 in FIGS. 3A and 3B), electrically associated with a high voltage power source (72 in FIG. 1B, 170 in FIGS. 1D, 2A, 2B, 3B and 4C), the electrode being configured to apply a plasma generating electromagnetic field within the vessel. The system further comprises an actuator (66, 152, 220, 290) configured to cause agitation of the liquid medium inside the vessel. And the system yet further comprises a chiller (230, 390, 400, 430, 470) having a cold portion (232, 392, 410, 446, 474, respectively) configured to thermally couple with the liquid medium inside the vessel. It is noted that any of the chillers may be used, mutatis mutandis, with the system of FIG. 1B. Chillers 230 and 390 may be used with the system of FIG. 2B. Chillers 400 and 430 may be used with the system of FIG. 4C. According to aspects of the invention, the system is configured to generate plasma at atmospheric pressure inside the vessel while the liquid medium is agitated and maintained at a temperature below room temperature.

According to some embodiments the liquid medium is a liquid composition comprising a biocompatible polymer, wherein the liquid medium freezes at temperatures lower than T1 and accepts a gel form at temperatures higher than T2, wherein T1<T2. According to some embodiments T1 is between about −5 degC. and about +20 degC., and T2 is between about +5 degC. and about +45 degC. According to some embodiments T1 is lower than about +15 degC., and T2 is higher than about +25 degC.

According to some embodiments the biocompatible polymer is synthetic.

According to some embodiments the chiller 230 or 390 may comprise a vapor-compression refrigerator. According to some embodiments the chiller comprises a vortex tube. According to some embodiments the chiller 230 or 390 comprises a thermoelectric cooler. According to some embodiments the chillers 400, 430 and 470 comprise an ice-pack (410, 444, 474 respectively) containing a coolant. According to some embodiments the chillers 400, 430 and 470 are detachable from the housing.

According to some embodiments the actuator (66 in FIG. 1B, 152 in FIG. 1D, 220 in FIGS. 2A, 3B and 4C, 290 in FIG. 2B) and the HV power source (72 in FIG. 1B, 170 in FIGS. 1D, 2A, 2B, 3B and 4C) are housed in a housing (62 in FIG. 1B, 130 in FIGS. 1D and 2A, 252 in FIG. 2B, 370 in FIG. 3B and 464 in FIG. 4C), and the vessel is detachable from the housing. According to some embodiments the walls of the vessel are dielectric. According to some embodiments the walls of the vessel are metallic. According to some embodiments the walls of the vessel are electrically connected to ground potential.

According to some embodiments the electrode is inside the vessel. According to some embodiments the electrode (38 in FIG. 1B, 172 in FIGS. 1D, 2A and 4C, 192 in FIG. 1E, 280 in FIG. 2B, and 320 in FIGS. 3A and 3B) is shaped to have a pointed tip and the plasma is generated in an arcing mode between the tip and the liquid medium. According to some embodiments the system further comprises a second electrode, electrically associated with a second high voltage power source, and configured to thereby apply a plasma generating electromagnetic field within the vessel.

According to some embodiments the actuator is a stirrer (220) and said agitation is affected by stirring the liquid medium. According to some embodiments the stirrer is a magnetic stirrer and the vessel contains a magnetic slab (222). According to some embodiments the actuator is a vibrating actuator (290) configured to vibrate and/or rotate the vessel thereby shaking the liquid medium. According to some embodiments the actuator is a compressor (66, 152) configured to force a stream of gas onto the liquid medium, thereby agitating the liquid medium. According to some embodiments the gas is released inside the liquid medium in the vessel, thereby generating bubbles in the liquid medium. In some embodiments two or more methods may be used together for agitating the liquid medium. For example, a compressor may be used to generate bubbles in the liquid while the liquid medium is stirred using a stirrer.

In some embodiments the electrode may be vibrated or displaced so that plasma is not generated over a single location of the liquid medium, thereby assisting in diminishing local heat accumulation in the liquid medium.

According to some embodiments the system (FIG. 1B, device 100, device 360) further comprising a compressor fluidly associated with the vessel via an inlet channel and a gas inlet port (34, 162, 330, respectively) of the vessel, the compressor being configured to force gas into the vessel. According to some embodiments, e.g., in FIGS. 1B and 3B the gas is air, drawn from the ambient. According to some embodiments, e.g., as demonstrated in FIG. 1D, the compressor draws the gas from the vessel via an outlet channel (154) fluidly associated with a gas outlet port (156) of the vessel, thereby circulating the gas through the vessel.

According to some embodiments, e.g., in FIGS. 1B, 1D, 1E and 3B, the plasma-generating EM field is applied to the stream of gas. According to some embodiments, the electrode (192, 320) is shaped as an open-ended tube fluidly associated with the compressor, the plasma-generating EM field is thereby generated along the stream of gas between the distal end of the tube and the liquid medium.

According to some embodiments, the inlet channel (e.g., slot port 70, inlet channels 160, 382), may comprise a filter. Such a filter in the inlet channel may be used to ensure sterility of the gas (e.g., air) that is forced into the vessel by filtering out bio-contaminants such as viruses or bacteria. According to some embodiments, the outlet channel (e.g., outlet port 350 may comprise a filter. Such a filter may be used to absorb or filter out hazardous residuals of the plasma, e.g., ozone, before releasing the gas to the ambient.

According to an aspect of the invention there is further provided a transportable closed vessel 300. The closed vessel comprises a liquid medium stored in the vessel, intended for medical use, and an electrode (320) electrically associated with a HV connector (340). The electrode is configured to apply a plasma-generating EM field inside the vessel upon receiving high voltage via the HV connector. The vessel is biologically sealed so as to prevent penetration of bacteria or viruses into the vessel. According to some embodiments, the vessel is hermetically sealed.

According to some embodiments, the liquid medium is sterile. According to some embodiments, the liquid medium comprises a biocompatible polymer, forming a liquid composition capable of undergoing a phase transition to a gel form upon a stimulus.

According to some embodiments the walls of the vessel are dielectric. According to some embodiments the walls of the vessel are metallic. According to some embodiments the vessel has an internal volume between 5 cc and 50 cc.

According to some embodiments the vessel further comprises an inlet port (330), configured to enable flowing a gas into the vessel.

According to some embodiments the electrode 320 comprises a metallic tube in flow communication with the inlet port and having a distal portion inside the vessel. According to some embodiments the metallic tube has a pointed tip at a distal end 322 thereof.

According to some embodiments the vessel further comprises a magnetic slab (222) immersed in the liquid medium and configured to cause stirring of the liquid when affected by a corresponding magnetic field.

According to some embodiments the vessel further comprises an outlet port (350), allowing flowing gas out of the vessel. According to some embodiments the vessel further comprises a removable cover (312), wherein the inlet port and HV connector are assembled onto the cover.

According to an aspect of the invention there is further provided a portable, passive chiller (400, 430, 470) configured to chill a vessel The passive chiller comprises an ice-pack (410, 444, 474, respectively) having at least one chamber containing a coolant. The coolant preferably has a freezing temperature between −10 degC. and +5 degC. The at least one chamber of the ice-pack is shaped to surround a slot (422, 442, 472, respectively) in the ice-pack, wherein the slot is configured and dimensioned to house the vessel therein.

According to some embodiments the passive chiller (e.g., chiller 400) further comprises the vessel (420) attached to the ice-pack in the slot, wherein the vessel is advantageously metallic and in direct contact with the coolant. According to some embodiments the vessel 420 comprises an openable cover to the vessel. According to some embodiments the vessel 420 further comprises a HV connector and an electrode electrically associated with the HV connector, as is depicted for example in vessel 300 in FIG. 3A. The electrode is preferably electrically insulated from the metallic vessel and configured to apply a plasma generating EM field in the vessel upon receiving high voltage from a HV power source.

According to some embodiments the slot of the passive chiller is a through-hole in the ice-pack. According to some embodiments the slot is shaped as a depression in the ice-pack. According to some embodiments the passive chiller (430) further comprises a non-rigid member surrounding the slot, configured to adjustably contact the vessel—thereby establishing thermal contact between the ice-pack and the vessel—when the vessel is placed in the slot. According to some embodiments the non-rigid member comprises a flexible metallic portion. According to some embodiments the non-rigid member comprises at least one second chamber 446, at least one wall thereof is non-rigid. The at least one second chamber preferably contains a material which is liquid at the freezing temperature of the coolant. According to some embodiments the at least one non-rigid wall is soft.

According to an aspect of the invention there is further provided a liquid composition comprising a biocompatible polymer, wherein the liquid composition undergoes a phase transition to a gel form upon a stimulus. The liquid composition further comprises the reactive species H₂O₂ and NO₂⁻ at a concentration of no less than 50 mg/liter, or no less than 100 mg/liter, or no less than 200 mg/liter wherein each possibility represents a separate embodiment.

According to an aspect of the invention there is further provided a liquid composition comprising a biocompatible polymer, wherein the liquid composition undergoes a phase transition to a gel form upon a stimulus. The liquid composition further comprises reactive species, at least one from the group consisting of OH*, HO₂*, O₂⁻, O₃, NO*, NO₃⁻, H₂O₂ NO₂⁻ and ONOO*, wherein the reactive species have been generated by atmospheric plasma in an arcing mode.

According to a further aspect of the invention there is provided a liquid composition comprising a synthetic biocompatible polymer, wherein the liquid composition undergoes a phase transition to a gel form upon a stimulus. The liquid composition further comprises reactive species, at least one from the group consisting of OH*, HO₂*, O₂⁻, O₃, NO*, NO₃⁻, H₂O₂, NO₂⁻ and ONOO*.

According to some embodiments the stimulus of any of the above-mentioned liquid compositions comprises a change in temperature. According to some embodiments the stimulus comprises a change in pH. According to some embodiments the stimulus comprises light, and the phase transition involves light-induced cross-linking.

According to some embodiments the synthetic biocompatible polymer comprises polyethylene glycol (PEG), polypropylene glycol (PPG), poly(meth)acrylic acid, poly (meth)acrylate or a combination thereof. According to some embodiments the synthetic biocompatible polymer comprises a poloxamer. According to some embodiments the poloxamer is selected from the group consisting of poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, or a mixture thereof.

According to some embodiments the liquid composition further comprises a fluid medium comprising a buffering or pH adjusting agent. According to some embodiments the buffering or pH adjusting agent is selected from the group consisting of 2-amino-2-hydroxymethyl-1,3-propanediol (Tris), 2-[bis(2-hydroxyethyl)imino]-2-(hydroxymethyl)-1,3-propanediol (bis-Tris), 4-morpholine ethane sulfonic acid (MES) buffer, ammonium chloride, bicine, tricine, sodium phosphate monobasic, sodium phosphate dibasic, sodium carbonate, sodium bicarbonate, sodium acetate, sodium phosphate, glutamic acid, citrate buffer, histidine buffer, Dulbecco's phosphate-buffered saline, 4-(2-hydroxyethyl)-1-piperazineethanesulfonic acid (HEPES), methoxypsoralen (MOPS), N-cyclohexyl-3-aminopropanesulfonic acid (CAPS), N-cyclohexyl-2-hydroxyl-3-aminopropanesulfonic acid (CAPSO), N-Cyclohexyl-2-aminoethanesulfonic acid (CHES), 3-[4-(2-Hydroxyethyl)-1-piperazinyl]propanesulfonic acid (HEPPS), phosphate-buffered saline, tris-buffered saline, Hank's solution, and Ringer's solution, and a mixture or combination thereof. According to some embodiments the liquid composition has a pH in the range of about 5.0 to about 7.5.

According to some embodiments the liquid composition may be used in treating a pre-malignant lesion. According to some embodiments the liquid composition may be used in treating a tumor. According to some embodiments the liquid composition may be used in preventing or delaying tumor recurrence following tumor excision. According to some embodiments the liquid composition may be used in treating an infection selected from a viral, a bacterial, a yeast, a mold, and a fungal infection.

According to a further aspect of the invention there is provided a foam comprising the reactive species H₂O₂ and NO₂ at a concentration no less than 50 mg/Kg, or no less than 100 mg/Kg, or no less than 200 mg/Kg wherein each possibility represents a separate embodiment. According to some embodiments the foam further comprises at least one of the reactive species OH*, HO₂*, O₂⁻, O₃, NO*, NO₃⁻, and ONOO*.

According to some embodiments the foam is made from a liquid composition that was plasma activated and transformed to foam by intense agitation. According to some embodiments the liquid composition comprises a biocompatible polymer, and is capable of undergoing a phase transition to a gel form upon a stimulus. According to some embodiments the stimulus comprises at least one from the group consisting of a change in temperature, a change in pH and a light-induced cross-linking.

According to a further aspect of the invention there is provided a method of treating an infection selected from a viral, a bacterial, a yeast, a mold, and a fungal infection in a subject in need thereof, the method comprising the step of topically administering to the subject a therapeutically effective amount of any of the liquid compositions described above or any of the foams generated therefrom.

According to some embodiments the method further comprises, prior to said step of topically administering, plasma activating the liquid composition, by forcing a stream of air onto a surface of the liquid composition while applying a plasma generating EM field between an electrode outside the liquid composition and the surface of the liquid composition, thereby generating plasma at ambient pressure in an arcing mode along the stream of air.

According to some embodiments the method further comprises agitating the liquid composition during said plasma activation. According to some embodiments the method yet further comprises maintaining the liquid composition at a temperature lower than room temperature during said plasma activation. According to some embodiments said temperature is lower than 10 degC.

According to a further aspect of the invention there is provided a method of treating a pre-malignant lesion in a subject in need thereof, the method comprising the step of contacting the lesion with a therapeutically effective amount of any of the liquid compositions described above or any of the foams generated therefrom.

According to a further aspect of the invention there is provided a method of treating a tumor in a subject in need thereof, the method comprising the step of contacting the tumor with a therapeutically effective amount of any of the liquid compositions described above or any of the foams generated therefrom.

According to a further aspect of the invention there is further provided a method of preventing or delaying tumor recurrence following tumor excision in a subject in need thereof, the method comprising the step of contacting the remaining tissue that surrounded the tumor with a therapeutically effective amount of any of the liquid compositions described above or any of the foams generated therefrom.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A system for providing plasma activated medium (PAM), the system comprising:
a vessel configured to contain a liquid medium;
an electrode electrically associated with a high voltage power source, configured to apply a plasma generating electromagnetic field within the vessel; and
an actuator configured to cause agitation of the liquid medium inside the vessel,
the system being configured to generate plasma at atmospheric pressure inside the vessel while the liquid medium is agitated.

2. The system of claim 1 wherein the liquid medium is a liquid composition comprising a biocompatible polymer, wherein the liquid medium freezes at temperatures lower than T1 and accepts a gel form at temperatures higher than T2, wherein T1<T2.

3. The system of claim 2 wherein T1 is between about −5 degC. and about +20 degC., and T2 is between about +5 degC. and about +45 degC.

4. (canceled)

5. (canceled)

6. The system of claim 78 wherein the chiller comprises a vapor-compression refrigerator.

7. The system of claim 6 wherein the chiller comprises a vortex tube.

8. The system of claim 78 wherein the chiller comprises a thermoelectric cooler or an ice-pack containing a coolant.

9. (canceled)

10. (canceled)

11. The system of claim 1 wherein the actuator and the HV power source are housed in a housing, and the vessel is detachable from the housing.

12. (canceled)

13. (canceled)

14. The system of claim 1 wherein the electrode is inside the vessel.

15. The system of claim 1 wherein the electrode is shaped to have a pointed tip and the plasma is generated in an arcing mode between the tip and the liquid medium.

16. The system of claim 1 further comprising a second electrode, electrically associated with a second high voltage power source, and configured to thereby apply a plasma generating electromagnetic field within the vessel.

17. The system of claim 1 wherein the actuator is a stirrer and said agitation is affected by stirring the liquid medium.

18. (canceled)

19. The system of claim 1 wherein the actuator is a vibrating actuator configured to vibrate the vessel thereby shaking the liquid medium.

20. The system of claim 1 wherein the actuator is a compressor configured to force a stream of gas onto the liquid medium, thereby agitating the liquid medium.

21. The system of claim 20 wherein the gas is released inside the liquid medium in the vessel, thereby generating bubbles in the liquid medium.

22. The system of claim 1 further comprising a compressor fluidly associated with the vessel via an inlet channel and a gas inlet port of the vessel, the compressor being configured to force gas into the vessel.

23. (canceled)

24. The system of claim 22 wherein the compressor draws the gas from the vessel via an outlet channel fluidly associated with a gas outlet port of the vessel, thereby circulating the gas through the vessel.

25. The system of claim 22 wherein the plasma-generating EM field is applied to the stream of gas.

26. The system of claim 25 wherein the electrode is shaped as an open-ended tube fluidly associated with the compressor, the plasma-generating EM field is thereby generated along the stream of gas between the distal end of the tube and the liquid medium.

27.-77. (canceled)

78. The system of claim 1 further comprising a chiller having a cold portion configured to thermally couple with the liquid medium inside the vessel, wherein the system is configured to generate plasma at atmospheric pressure inside the vessel while the liquid medium is maintained at a temperature below room temperature.

79. The system of claim 1 wherein the PAM is provided for medical use.
